(19) United States
(12) Patent Application Publication
(10) Pub. No.: US 2002/0098511 A1
(43) Pub. Date: Jul. 25, 2002

(54) Protein-protein interactions

(76) Inventors: Karen Heichman, Salt Lake City, UT (US); Daniel M. Cimbora, Salt Lake City, UT (US); Paul L. Bartel, Salt Lake City, UT (US)

Correspondence Address: ROTHWELL, FIGG, ERNST & MANBECK, P.C., 555 13TH STREET, N.W., SUITE 701, EAST TOWER, WASHINGTON, DC, 20004, US

(21) Appl. No.: 09/727,384

(22) Filed: Dec. 1, 2000

Related U.S. Application Data
(60) Provisional application No. 60/168,377, filed on Dec. 2, 1999.
(60) Provisional application No. 60/168,379, filed on Dec. 2, 1999.
(60) Provisional application No. 60/185,056, filed on Feb. 25, 2000.

(51) Int. Cl. A01K 67/00; G01N 33/53; C12N 5/06

(52) U.S. Cl. 435/7.1; 800/9; 435/325; 530/388.1

(57) ABSTRACT

The present invention relates to the discovery of novel protein-protein interactions that are involved in mammalian physiological pathways, including physiological disorders or diseases. Examples of physiological disorders and diseases include non-insulin dependent diabetes mellitus (NIDDM), neurodegenerative disorders, such as Alzheimer's Disease (AD), and the like. Thus, the present invention is directed to complexes of these proteins and/or their fragments, antibodies to the complexes, diagnosis of physiological generative disorders (including diagnosis of a predisposition to and diagnosis of the existence of the disorder), drug screening for agents which modulate the interaction of proteins described herein, and identification of additional proteins in the pathway common to the proteins described herein.

### CROSS-REFERENCE TO RELATED APPLICATIONS

The present application is related to U.S. provisional patent applications Serial No. 60/168,377 filed Dec. 2, 1999, Serial No. 60/168,379 filed Dec. 2, 1999 and Serial No. 60/185,056 filed Feb. 25, 2000, each incorporated herein by reference.

### BACKGROUND OF THE INVENTION

The present invention relates to the discovery of novel protein-protein interactions that are involved in mammalian physiological pathways, including physiological disorders or diseases.

Examples of physiological disorders and diseases include non-insulin dependent diabetes mellitus (NIDDM), neurodegenerative disorders, such as Alzheimer's Disease (AD), and the like. Thus, the present invention is directed to complexes of these proteins and/or their fragments, antibodies to the complexes, diagnosis of physiological generative disorders (including diagnosis of a predisposition to and diagnosis of the existence of the disorder), drug screening for agents which modulate the interaction of proteins described herein, and identification of additional proteins in the pathway common to the proteins described herein.

The publications and other materials used herein to illuminate the background of the invention, and in particular, cases to provide additional details respecting the practice, are incorporated herein by reference, and for convenience, are referenced by author and date in the following text and respectively grouped in the appended List of References.

Many processes in biology, including transcription, translation and metabolic or signal transduction pathways, are mediated by non-covalently associated protein complexes. The formation of protein-protein complexes or protein-DNA complexes produce the most efficient chemical machinery. Much of modern biological research is concerned with identifying proteins involved in cellular processes, determining their functions, and how, when and where they interact with other proteins involved in specific pathways. Further, with rapid advances in genome sequencing, there is a need to define protein linkage maps, i.e., detailed inventories of protein interactions that make up functional assemblies of proteins or protein complexes or that make up physiological pathways.

Recent advances in human genomics research has led to rapid progress in the identification of novel genes. In applications to biological and pharmaceutical research, there is a need to determine functions of gene products. A first step in defining the function of a novel gene is to determine its interactions with other gene products in appropriate context. That is, since proteins make specific interactions with other proteins or other biopolymers as part of functional assemblies or physiological pathways, an appropriate way to examine function of a gene is to determine its physical relationship with other genes. Several systems exist for identifying protein interactions and hence relationships between genes.

There continues to be a need in the art for the discovery of additional protein-protein interactions involved in mammalian physiological pathways. There continues to be a need in the art also to identify the protein-protein interactions that are involved in mammalian physiological disorders and diseases, and to thus identify drug targets.

### SUMMARY OF THE INVENTION

The present invention relates to the discovery of protein-protein interactions that are involved in mammalian physiological pathways, including physiological disorders or diseases, and to the use of this discovery. The identification of the interacting proteins described herein provide new targets for the identification of useful pharmaceuticals, new targets for diagnostic tools in the identification of individuals at risk, sequences for production of transformed cell lines, cellular models and animal models, and new bases for therapeutic intervention in such physiological pathways Thus, one aspect of the present invention is protein complexes. The protein complexes are a complex of (a) two interacting proteins, (b) a first interacting protein and a fragment of a second interacting protein, (c) a fragment of a first interacting protein and a second interacting protein, or (d) a fragment of a first interacting protein and a fragment of a second interacting protein. The fragments of the interacting proteins include those parts of the proteins, which interact to form a complex. This aspect of the invention includes the detection of protein interactions and the production of proteins by recombinant techniques. The latter embodiment also includes cloned sequences, vectors, transfected or transformed host cells and transgenic animals.

A second aspect of the present invention is an antibody that is immunoreactive with the above complex. The antibody may be a polyclonal antibody or a monoclonal antibody. While the antibody is immunoreactive with the complex, it is not immunoreactive with the component parts of the complex. That is, the antibody is not immunoreactive with a first interactive protein, a fragment of a first interacting protein, a second interacting protein or a fragment of a second interacting protein. Such antibodies can be used to detect the presence or absence of the protein complexes.

A third aspect of the present invention is a method for diagnosing a predisposition for physiological disorders or diseases in a human or other animal. The diagnosis of such disorders includes a diagnosis of a predisposition to the disorders and a diagnosis for the existence of the disorders. In accordance with this method, the ability of a first interacting protein or fragment thereof to form a complex with a second interacting protein or a fragment thereof is assayed, or the genes encoding interacting proteins are screened for mutations in interacting portions of the protein molecules. The inability of a first interacting protein or fragment thereof to form a complex, or the presence of mutations in a gene within the interacting domain, is indicative of a predisposition to, or existence of a disorder. In accordance with one embodiment of the invention, the ability to form a complex is assayed in a two-hybrid assay. In a first aspect of this embodiment, the ability to form a complex is assayed by a yeast two-hybrid assay. In a second aspect, the ability to form a complex is assayed by a mammalian two-hybrid assay. In a second embodiment, the ability to form a complex is assayed by measuring in vitro a complex formed by combining said first protein and said second protein. In one aspect the proteins are isolated from a human or other animal. In a third embodiment, the ability to form a complex is assayed by measuring the binding of an antibody, which is specific for the complex. In a fourth embodiment, the ability to form a complex is assayed by measuring the binding of an antibody that is specific for the complex with a tissue extract from a human or other animal. In a fifth embodiment, coding sequences of the interacting proteins described herein are screened for mutations.

A fourth aspect of the present invention is a method for screening for drug candidates which are capable of modulating the interaction of a first interacting protein and a second interacting protein. In this method, the amount of the complex formed in the presence of a drug is compared with the amount of the complex formed in the absence of the drug. If the amount of complex formed in the presence of the drug is greater than or less than the amount of complex formed in the absence of the drug, the drug is a candidate for modulating the interaction of the first and second interacting proteins. The drug promotes the interaction if the complex formed in the presence of the drug is greater and inhibits (or disrupts) the interaction if the complex formed in the presence of the drug is less. The drug may affect the interaction directly, i.e., by modulating the binding of the two proteins, or indirectly, e.g., by modulating the expression of one or both of the proteins.

A fifth aspect of the present invention is a model for such physiological pathways, disorders or diseases. The model may be a cellular model or an animal model, as further described herein. In accordance with one embodiment of the invention, an animal model is prepared by creating transgenic or “knock-out” animals. The knock-out may be a total knock-out, i.e., the desired gene is deleted, or a conditional knock-out, i.e., the gene is active until it is knocked out at a determined time. In a second embodiment, a cell line is derived from such animals for use as a model. In a third embodiment, an animal model is prepared in which the biological activity of a protein complex of the present invention has been altered. In one aspect, the biological activity is altered by disrupting the formation of the protein complex, such as by the binding of an antibody or small molecule to one of the proteins which prevents the formation of the protein complex. In a second aspect, the biological activity of a protein complex is altered by disrupting the action of the complex, such as by the binding of an antibody or small molecule to the protein complex which interferes with the action of the protein complex as described herein. In a fourth embodiment, a cell model is prepared by altering the genome of the cells in a cell line. In one aspect, the genome of the cells is modified to produce at least one protein complex described herein. In a second aspect, the genome of the cells is modified to eliminate at least one protein of the protein complexes described herein.

A sixth aspect of the present invention are nucleic acids coding for novel proteins discovered in accordance with the present invention and the corresponding proteins and antibodies.

A seventh aspect of the present invention is a method of screening for drug candidates useful for treating a physiological disorder. In this embodiment, drugs are screened on the basis of the association of a protein with a particular physiological disorder. This association is established in accordance with the present invention by identifying a relationship of the protein with a particular physiological disorder. The drugs are screened by comparing the activity of the protein in the presence and absence of the drug. If a difference in activity is found, then the drug is a drug candidate for the physiological disorder. The activity of the protein can be assayed in vitro or in vivo using conventional techniques, including transgenic animals and cell lines of the present invention.

### DETAILED DESCRIPTION OF THE INVENTION

The present invention is the discovery of novel interactions between proteins described herein. The genes coding for some of these proteins may have been cloned previously, but their potential interaction in a physiological pathway or with a particular protein was unknown. Alternatively, the genes coding for some of these proteins have not been cloned previously and represent novel genes. These proteins are identified using the yeast two-hybrid method and searching a human total brain library, as more filly described below.

According to the present invention, new protein-protein interactions have been discovered. The discovery of these interactions has identified several protein complexes for each protein-protein interaction. The protein complexes for these interactions are set forth below in Tables 1-31, which also identify the new protein-protein interactions of the present invention.

TABLE 1
Protein Complexes of p38 alpha/CYT4 Interaction
Protein Kinase p38 alpha (p38 alpha) and CYT4
A fragment of p38 alpha and CYT4
p38 alpha and a fragment of CYT4
A fragment of p38 alpha and a fragment of CYT4

TABLE 2
Protein Complexes of MAPKAP-K3/PN2012 Interaction
MAP Kinase MAPKAP-K3 (MAPKAP-K3) and
Novel Protein PN2012 (PN2012)
A fragment of MAPKAP-K3 and PN2012
MAPKAP-K3 and a fragment of PN2012
A fragment of MAPKAP-K3 and a fragment of PN2012

TABLE 3
Protein Complexes of MAPKAP-K3/PN7771 Interaction
MAP Kinase MAPKAP-K3 (MAPKAP-K3) and
Novel Protein Fragment PN7771 (PN7771)
A fragment of MAPKAP-K3 and PN7771
MAPKAP-K3 and a fragment of PN7771
A fragment of MAPKAP-K3 and a fragment of PN7771

TABLE 4
Protein Complexes of PRAK/PN7098 Interaction
Protein Kinase PRAK (PRAK) and
Novel Protein Fragment PN7098 (PN7098)
A fragment of PRAK and PN7098
PRAK and a fragment of PN7098
A fragment of PRAK and a fragment of PN7098

TABLE 5
Protein Complexes of PRAK/Kendrin Interaction
Protein kinase PRAK (PRAK) and kendrin
A fragment of PRAK and kendrin
PRAK and a fragment of kendrin
A fragment of PRAK and a fragment of kendrin

TABLE 6
Protein Complexes of PRAK/Homeotic Protein Prox1 Interaction
Protein kinase PRAK (PRAK) and Homeotic Protein Prox1 (Prox 1)
A fragment of PRAK and Prox1
PRAK and a fragment of Prox1
A fragment of PRAK and a fragment of Prox1

TABLE 7
Protein Complexes of PRAK/Hook1 Interaction
Protein kinase PRAK (PRAK) and Hook1
A fragment of PRAK and Hook1
PRAK and a fragment of Hook1
A fragment of PRAK and a fragment of Hook1

TABLE 8
Protein Complexes of PRAK/IG Heavy Chain Constant Region Interaction
Protein kinase PRAK (PRAK) and IG heavy chain constant region
A fragment of PRAK and IG heavy chain constant region
PRAK and a fragment of IG heavy chain constant region
A fragment of PRAK and a fragment of IG heavy chain constant region

TABLE 9
Protein Complexes of PRAK/Golgin-95 Interaction
Protein kinase PRAK (PRAK) and golgin-95
A fragment of PRAK and golgin-95
PRAK and a fragment of golgin-95
A fragment of PRAK and a fragment of golgin-95

TABLE 10
Protein Complexes of PRAK/KIAA0555 Interaction
Protein kinase PRAK (PRAK) and KIAA0555
A fragment of PRAK and KIAA0555
PRAK and a fragment of KIAA0555
A fragment of PRAK and a fragment of KIAA0555

TABLE 11
Protein Complexes of PRAK/Leucine-rich Protein L130 Interaction
Protein kinase PRAK (PRAK) and leucine-rich protein L130
A fragment of PRAK and leucine-rich protein L130
PRAK and a fragment of leucine-rich protein L130
A fragment of PRAK and a fragment of leucine-rich protein L130

TABLE 12
Protein Complexes of PRAK/ERK3 Interaction
Protein kinase PRAK (PRAK) and ERK3
A fragment of PRAK and ERK3
PRAK and a fragment of ERK3
A fragment of PRAK and a fragment of ERK3

TABLE 13
Protein Complexes of PRAK/cAMP-dependent Protein Kinase Interaction
Protein kinase PRAK (PRAK) and cAMP-dependent protein kinase
A fragment of PRAK and cAMP-dependent protein kinase
PRAK and a fragment of cAMP-dependent protein kinase
A fragment of PRAK and a fragment of cAMP-dependent protein kinase

TABLE 14
Protein Complexes of PRAK/AL117538
Protein kinase PRAK (PRAK) and AL117538
A fragment of PRAK and AL117538
PRAK and a fragment of AL117538
A fragment of PRAK and a fragment of AL117538

TABLE 15
Protein Complexes of PRAK/AL117237
Protein kinase PRAK (PRAK) and AL117237
A fragment of PRAK and AL117237
PRAK and a fragment of AL117237
A fragment of PRAK and a fragment of AL117237

TABLE 16
Protein Complexes of p38 Alpha/JNK3 Alpha2 Interaction
Protein Kinase p38 alpha (p38 alpha) and JNK3 alpha2
A fragment of p38 alpha and JNK3 alpha2
p38 alpha and a fragment of JNK3 alpha2
A fragment of p38 alpha and a fragment of JNK3 alpha2

TABLE 17
Protein Complexes p38 Alpha/C-Nap1 Interaction
Protein Kinase p38 alpha (p38 alpha) and C-Nap1
A fragment of p38 alpha and C-Nap1
p38 alpha and a fragment of C-Nap1
A fragment of p38 alpha and a fragment of C-Nap1

TABLE 18
Protein Complexes p38 Alpha/Vinculin Interaction
Protein Kinase p38 alpha (p38 alpha) and Vinculin
A fragment of p38 alpha and Vinculin
p38 alpha and a fragment of Vinculin
A fragment of p38 alpha and a fragment of Vinculin

TABLE 19
Protein Complexes p38 Alpha K53M Mutant/
Splicing Factor PSF Interaction
Protein Kinase p38 alpha (p38 alpha) K53M Mutant and
Splicing Factor PSF
A fragment of p38 alpha K53M Mutant and Splicing Factor PSF
p38 alpha K53M Mutant and a fragment of Splicing Factor PSF
A fragment of p38 alpha K53M Mutant and
a fragment of Splicing Factor PSF

TABLE 20
Protein Complexes of MAPKAP-K2/Leucine-rich Protein L130 Interaction
MAPKAP-K2 and leucine-rich protein L130
A fragment of MAPKAP-K2 and leucine-rich protein L130
MAPKAP-K2 and a fragment of leucine-rich protein L130
A fragment of MAPKAP-K2 and a fragment of leucine-rich protein L130

TABLE 21
Protein Complexes of MAPKAP-K2/
cAMP-dependent Protein Kinase Interaction
MAPKAP-K2 and cAMP-dependent Protein Kinase
A fragment of MAPKAP-K2 and cAMP-dependent Protein Kinase
MAPKAP-K2 and a fragment of cAMP-dependent Protein Kinase
A fragment of MAPKAP-K2 and a cAMP-dependent Protein Kinase

TABLE 22
Protein Complexes of MAPKAP-K2/SET Interaction
MAPKAP-K2 and SET
A fragment of MAPKAP-K2 and SET
MAPKAP-K2 and a fragment of SET
A fragment of MAPKAP-K2 and a SET

TABLE 23
Protein Complexes of MAPKAP-K2/TL21 Interaction
MAPKAP-K2 and TL21
A fragment of MAPKAP-K2 and TL21
MAPKAP-K2 and a fragment of TL21
A fragment of MAPKAP-K2 and a TL21

TABLE 24
Protein Complexes of MAPKAP-K2
(K93M, T222D, T334D Mutant)/ERK3 Interaction
MAPKAP-K2 K93M, T222D, T334D Mutant and ERK3
A fragment of MAPKAP-K2 K93M, T222D, T334D Mutant and ERK3
MAPKAP-K2 K93M, T222D, T334D Mutant and a fragment of ERK3
A fragment of MAPKAP-K2 K93M, T222D, T334D Mutant and a ERK3

TABLE 25
Protein Complexes of MAPKAP-K3/Thrombospondin 3 Interaction
MAPKAP-K3 and thrombospondin 3
A fragment of MAPKAP-K3 and thrombospondin 3
MAPKAP-K3 and a fragment of thrombospondin 3
A fragment of MAPKAP-K3 and a fragment of thrombospondin 3

TABLE 26
Protein Complexes of MAPKAP-K3/Malate Dehydrogenase Interaction
MAPKAP-K3 and malate dehyrdrogenase
A fragment of MAPKAP-K3 and malate dehyrdrogenase
MAPKAP-K3 and a fragment of malate dehyrdrogenase
A fragment of MAPKAP-K3 and a fragment of malate dehyrdrogenase

TABLE 27
Protein Complexes of MAPKAP-K3/GA17 Interaction
MAPKAP-K3 and GA17
A fragment of MAPKAP-K3 and GA17
MAPKAP-K3 and a fragment of GA17
A fragment of MAPKAP-K3 and a fragment of GA17

TABLE 28
Protein Complexes of MAPKAP-K3/Calpain 4 Small Subunit Interaction
MAPKAP-K3 and Calpain 4 small subunit
A fragment of MAPKAP-K3 and Calpain 4 small subunit
MAPKAP-K3 and a fragment of Calpain 4 small subunit
A fragment of MAPKAP-K3 and a fragment of Calpain 4 small subunit

TABLE 29
Protein Complexes of MAPKAP-K3/BAT3 Interaction
MAPKAP-K3 and BAT3
A fragment of MAPKAP-K3 and BAT3
MAPKAP-K3 and a fragment of BAT3
A fragment of MAPKAP-K3 and a fragment of BAT3

TABLE 30
Protein Complexes of MSK-1/AbLim Interaction
MSK-1 and abLim
A fragment of MSK-1 and abLim
MSK-1 and a fragment of abLim
A fragment of MSK-1 and a fragment of abLim

TABLE 31
Protein Complexes of MSK-1/KIAA0144 Interaction

MSK-1 and KIAA0144
A fragment of MSK-1 and KIAA0144
MSK-1 and a fragment of KIAA0144
A fragment of MSK-1 and a fragment of KIAA0144

The involvement of above interactions in particular pathways is as follows.

Many cellular proteins exert their function by interacting with other proteins in the cell. Examples of this are found in the formation of multiprotein complexes and the association of an enzymes with their substrates. It is widely believed that a great deal of information can be gained by understanding individual protein-protein interactions, and that this is useful in identifying complex networks of interacting proteins that participate in the workings of normal cellular functions. Ultimately, the knowledge gained by characterizing these networks can lead to valuable insight into the causes of human diseases and can eventually lead to the development of therapeutic strategies. The yeast two-hybrid assay is a powerful tool for determining protein-protein interactions and it has been successfully used for studying human disease pathways. In one variation of this technique, a protein of interest (or a portion of that protein) is expressed in a population of yeast cells that collectively contain all protein sequences. Yeast cells that possess protein sequences that interact with the protein of interest are then genetically selected and the identity of those interacting proteins are determined by DNA sequencing. Thus, proteins that can be demonstrated to interact with a protein known to be involved in a human disease are therefore also implicated in that disease. To create a more complex network of interactions in a disease pathway, proteins that were identified in the first round of two-hybrid screening are subsequently used in two-hybrid assays as the protein of interest.

Cellular events that are initiated by exposure to growth factors, cytokines and stress are propagated from the outside of the cell to the nucleus by means of several protein kinase signal transduction cascades. p38 kinase is a member of the MAP kinase family of protein kinases. It is a key player in signal transduction pathways induced by the proinflammatory cytokines such as tumor necrosis factor (TNF), interleukin-1 (IL-1) and interleukin-6 (IL-6) and it also plays a critical role in the synthesis and release of the proinflammatory cytokines (Raingeaud et al., 1995; Lee et al., 1996; Miyazawa et al., 1998; Lee et al., 1994). Studies of inhibitors of p38 kinase have shown that blocking p38 kinase activity can cause anti-inflammatory effects, thus suggesting that this may be a mechanism of treating certain inflammatory diseases such as rheumatoid arthritis and inflammatory bowel disease. Further, p38 kinase activity has been implicated in other human diseases such as atherosclerosis, cardiac hypertrophy and hypoxic brain injury (Grammner et al., 1998; Mach et al., 1998; Wang et al., 1998; Nemoto et al., 1998; Kawasaki et al., 1997). Thus, by understanding p38 function, one may gain novel insight into a cellular response mechanism that affects a number of tissues and can potentially lead to harmful affects when disrupted.

The search for the physiological substrates of p38 kinase has taken a number of approaches including a variety of biochemical and cell biological methods. There are four known human isoforms of p38 kinase termed alpha, beta, gamma and delta, and these are thought to possess different physiological functions, likely because they have distinct substrate and tissue specificities.

Some of the p38 kinase substrates are known, and the list includes transcription factors and additional protein kinases that act downstream of p38 kinase. Four of the kinases that act downstream of p38 kinase, MAPKAP-K2, MAPKAP-K3, PRAK and MSK1, are currently being analyzed themselves and some of their substrates and regulators have been identified.

Initial two-hybrid screens have been performed and the search results are now described.

The yeast two-hybrid system has been used to detect potential substrates and upstream regulators of the p38 kinases and their downstream kinases. In a two-hybrid search using p38 alpha as the protein of interest, an interaction with the guanine nucleotide-exchange protein cytohesin-4 (CYT4) was identified. CYT4 is a member of the PSCD protein family and has a structural organization identical to other PSCD proteins, consisting of an N-terminal coiled-coil motif, a central Sec7 homology domain, and a C-terminal pleckstrin homology (PH) domain. The coiled-coil motif is involved in homodimerization, the Sec7 domain contains guanine-nucleotide exchange protein (GEP) activity, and the PH domain interacts with phospholipids and is responsible for association of PSCD proteins with membranes. Members of this family appear to mediate the regulation of protein sorting and membrane trafficking. CYT4 exhibits GEP activity in vitro with ADP-ribosylation factors ARF1 and ARF5 but is inactive with ARF6 (Ogasawara et al., 2000). CYT4 may act as either a substrate or a regulator of p38 alpha kinase in inflammation or other disease-related signal transduction pathways.

When the mitogen-activated MAP kinase activator 3pK (MAPKAP-K3) was used in a two-hybrid search, two interactors were identified. The first novel protein, PN2012, bears similarity to the mouse transcription factor Kaiso (GenBank accession AF097416). Kaiso, is a zinc-finger containing protein of the POZ-ZF variety; other related members of this family have been implicated in developmental control and cancer (Daniel et al., 1999). MAPKAP-K3 may phosphorylate this putative transcription factor, thereby altering its activity and affecting the transcription of a set of inflammation-related genes. In support of this hypothesis, Kaiso contains one MAPKAP consensus phosphorylation site.

The second interactor identified for MAPKAP-K3 is the novel protein PN7771. PN7771 is highly related (greater than 90% amino acid identity) to Ninein. Ninein is a centrosome-associated protein that interacts with human glycogen synthase kinase 3beta (GSK-3beta) (Hong et al., 2000), is localized to the pericentriolar matrix of the centrosome, and reacts with centrosomal autoantibody sera (Mack et al., 1998). PN7771 contains predicted calcium-binding EF hand motifs, a potential nuclear localization signal, a basic region-leucine zipper motif, a spectrin repeat, coiled-coil motifs, and Glu- and Gln-rich regions. The interaction with MAPKAP-K3 suggests PN7771 may be responsive to MAPK signaling pathways, perhaps serving as a substrate for MAPKAP-K3.

In support of this, we find several MAPKAP consensus phosphorylation sites in PN7771.

In a two-hybrid search using the p38-regulated protein kinase PRAK, an interaction with the novel protein PN7098 was identified. PN7098 is a 1,231 amino acid polypeptide, although the sequence is incomplete at the 3′ (C-terminal) end. PN7098 contains a PKC C1 (diacylglycerol/phorbol ester-binding) domain, several Ser-rich regions, and two potential nuclear localization signals. PN7098 is related (86% amino acid identity) to the rat Munc13-3 protein (GenBank accession U75361), which is involved in neurotransmitter release (Augustin, et al., 1999) PN7098 may function as either a regulator or a substrate of PRAK protein kinase activity.

Further two-hybrid screens have also been performed and the search results are now described. In a two-hybrid search using p38 alpha kinase as the protein of interest, four proteins were shown to bind to p38 alpha. The first protein, JNK3 alpha2, is also a serine/threonine protein kinase of the MAP kinase family that is involved in signal transduction (Gupta et al., 1996). Like the p38 kinase pathway constituents, the JNK kinases are activated in response to extracellular stimulation by IL-1. The JNK kinases function by phosphorylating various transcription factors, thereby altering gene expression patterns. The interaction of p38 alpha and JNK3 alpha2 suggests that JNK3 alpha2 is either a substrate or a regulator of p38 alpha, and further identifies a potential link between JNK3 and the inflammatory response. Is further support of such a link, we have subsequently identified yeast two-hybrid interactions between p38 alpha and both JNK1 and JNK2.

The second protein that interacts with p38 alpha is the large centrosomal protein C-NAP 1. C-NAP1 is a 2,442 amino acid protein that was originally identified by its interaction with the Nek2 cell cycle-regulated protein kinase (Fry et al., 1998). C-NAP1 contains multiple coiled-coiled domains that are likely to be involved in protein-protein interactions. The finding that C-NAP1 interacts with p38 alpha suggests that it is a substrate of both Nek2 and p38 kinases. Thus, C-NAP1 may play a critical role in cellular growth control and in the cellular inflammatory response. Further, by inference, this result links p38 alpha to cellular growth control and Nek2 to inflammation.

The third p38 alpha-interacting protein, vinculin, resides in the cytoplasmic side of adhesion plaques and may participate in actin microfilament attachment (Rudiger, 1998). Vinculin has been characterized as a tumor suppressor, suggesting that it may play a regulatory function in addition to a structural role in the cell. Vinculin is post-translationally modified by phosphorylation, suggesting it may be a substrate for p38 kinase. Given the requirements for cytoskeletal rearrangement and changes in cell adhesion in the inflammatory response, our results suggest that phosphorylation of vinculin by p38 alpha may be involved in cellular responses to inflammatory stimuli. This interaction is reminiscent of another interaction (see below) between a kinase downstream of p38 (MSK1) and the actin-binding protein ABLIM.

The fourth p38 alpha-interacting protein was identified with a mutant p38 alpha, in which lysine 53 was changed to a methionine (K53M), rendering the kinase catalytically inactive and presumably stabilizing transient protein-protein interactions. Using this K53M mutant as bait in a two-hybrid assay, the RNA splicing factor PSF was found to be an interactor. PSF is a nuclear protein that contains two RNA recognition motifs and has been found to form a complex with the polypyrimidine tract-binding protein PTB (Patton et al., 1993). Regulation of mRNA splicing is an effective way to modulate protein expression levels, and consequently the interaction of PSF and p38 alpha suggests that phosphorylation of the former by the latter may result in changes in the expression of proteins involved in the inflammatory response. Interestingly, PSF has been shown to bind to the protein phosphatase PP1 delta (Hirano et al., 1996), suggesting a scenario in which PSF activity is controlled by the opposite actions of p38 alpha kinase and PP1 delta phosphatase.

MAPKAP-K2, a protein kinase that acts downstream of p38 kinase in the same signal transduction pathway, was used in a two-hybrid search to identify potential substrates or regulators. MAPKAP-K2 was demonstrated to interact with five proteins. The first of these is a leucine-rich protein L130. L130 was identified by virtue of its high level of expression in hepatoblastoma cells (Hou et al., 1994). The expression of L 130 in hepatoblastoma cells suggests a role in liver function or in the transformation of normal cells to malignant ones. Interestingly, this protein was also identified as a two-hybrid interactor of another highly related p38-activated protein kinase, PRAK (see below). L130 interacts with the kinase domains of both MAPKAP-K2 and PRAK, suggesting it is a substrate for these kinases. Furthermore, the identification of L130 as an interactor of two kinases involved in the same signaling pathway strongly suggests an important role for L130 in the inflammatory response.

The second MAPKAP-K2 interactor, cAMP-dependent protein kinase (PKA) regulatory subunit type I alpha, is one component of the PKA serine/threonine protein kinase complex that plays a role in cellular signal transduction. Intracellular levels of cAMP increase in response to various chemical and hormonal stimuli, and PKA is in turn activated by binding to the second messenger cAMP (Francis et al., 1999). The regulatory subunit of PKA is phosphorylated, suggesting PKA may serve as a substrate for MAPKAP-K2. Consistent with this, the region of MAPKAP-K2 that interacts with PKA includes the kinase domain. In addition, we find that that this same subunit of PKA can bind to another p38-activated protein kinase, PRAK (see below). Although the region of PRAK with which PKA interacts does not include the kinase domain, this region of PRAK also interacts with ERK3, another kinase involved in signal transduction. Interestingly, ERK3 also interacts directly with MAPKAP-K2 (see below). Taken together, these results argue that PKA may be involved in the inflammatory response, perhaps as a substrate of these protein kinases.

Another MAPKAP-K2 interactor involved in signal transduction, ERK3, was found using the MAPKAP-K2 K93M, T222D, T334D triple mutant protein as bait. ERK3 (extracellular signal-regulated protein kinase 3) is a serine/threonine protein kinase (Cheng et al., 1996). It is a nuclear protein presnt in several tissues and is expressed in response to a number of extracellular stimuli.

Although the biological roles of ERK3 are not yet well understood, it is likely to be part of the MAP kinase cascade initiated in response to pro-inflammatory stimuli. This role is further supported by its interaction with the p38-regulated kinase PRAK; the interactions of ERK3 with both MAPKAP-K2 and PRAK have been confirmed by in vitro assays (see below).

Another signal transduction protein that binds MAPKAP-K2 is the myeloid leukemia-associated protein SET. SET may be involved in the generation of intracellular signaling events that lead to changes in transcriptional activity after binding of a ligand to HLA class II molecules (Vaesen et al., 1994). In addition, SET is a strong inhibitor of protein phosphatase 2A (Li et al., 1996). Furthermore, SET appears to play a role in cell proliferation, as SET mRNA expression is markedly reduced in cells rendered quiescent by serum starvation, contact inhibition, or differentiation (Carlson et al., 1998). Consistent with a role for SET in growth control and differentiation, fusion of the SET protein with part of the CAN oncogene as the result of a chromosome translocation results in leukemia (von Lindern et al., 1992). SET is a ubiquitously expressed nuclear phosphoprotein that resembles members of the nucleosome assembly protein family. The SET protein is phosphorylated on serine and threonine residues (in addition to tyrosines), suggesting SET may be a substrate of MAPKAP-K2 kinase activity.

The fourth MAPKAP-K2 interactor is the protein product of the TL21 transcript. In a study designed to examine cDNAs that are differentially expressed between androgen-dependent and androgen-independent prostate carcinoma cell lines, TL21 was isolated as a transcript showing a marked increase in the androgen-dependent cell line (Blok et al., 1995). The TL21 protein product with which MAPKAP-K2 interacts contains no discernible structural motifs, and consequently possible functions of TL21 cannot be deduced. However, the interaction with MAPKAP-K2 suggests it may serve as a substrate or regulator of MAPKAP-K2 kinase activity.

When a second p38-activated protein kinase, MAPKAP-K3, was used in a two-hybrid search, five proteins were demonstrated to interact with it. The first MAPKAP-K3 interactor is thrombospondin 3, an adhesive glycoprotein that is involved in cell-to-cell and cell-to-matrix interactions (Qabar et al., 1994). It is normally localized extracellularly; however, a number of extracellular proteins exist at low concentrations, or in certain cell types, within the cytoplasm, so we cannot rule out a biological role for the interaction with MAPKAP-K3 in the inflammatory response.

The second MAPKAP-K3 interactor is malate dehydrogenase, a cytoplasmic enzyme that catalyzes an NAD-dependent reversible reaction of the citric acid cycle (Musrati et al., 1998). The finding that MAPKAP-K3 interacts with this protein suggests the protein kinase cascade that responds to inflammatory stimuli may affect cellular metabolism.

The third MAPKAP-K3-interacting protein, GA17, has no known function; it is described in the public databases only as a novel gene isolated from human dendritic cells. The only discernible structural feature is a PCI or PINT domain near the C-terminus; this domain is found in proteasome subunits and proteins involved in translation initiation and intracellular signal transduction, but it has no known function. Although functions of this protein are not yet apparent, we infer that it may serve either upstream or downstream of MAPKAP-K3 in the inflammation response pathway.

The fourth MAPKAP-K3 interactor is the small subunit of the calcium-dependent protease calpain. Calpain is a non-lysosomal calcium-activated thiol-protease composed of large and small subunits; the small subunit with which MAPKAP-K3 interacts possesses regulatory activity. The true biological substrates of calpain are unknown, however a multitude of proteins can act as substrates in vitro (Saido et al., 1994). Interestingly, calpain has been shown to interact with IL-2 receptor gamma chain, and is responsible for cleavage of this protein (Noguchi et al., 1997).

Furthermore, calpain inhibitors have been shown to interfere with NFKB activation (Kouba et al., 2000), further implicating calpain in intracellular signaling in response to external stimuli. In light of these results, the interaction with MAPKAP-K3 suggests calpain activity may be modulated by MAPKAP-K3 phosphorylation, and that this has an effect on signal transduction in response to inflammatory signals.

The fifth MAPKAP-K3-interacting protein is BAT3. BAT3 a large proline-rich protein of unknown function that was identified as an HLA-B-associated transcript and was cloned from a human T-cell line (Banedi et al., 1990). BAT3 is a large cytoplasmic protein that is very rich in proline and includes short tracts of polyproline, polyglycine, and charged amino acids. BAT3 transcripts are present in all adult tissues with the highest levels found in testis (Ozaki et al., 1999). BAT3 was demonstrated to bind to a candidate neuroblastoma tumor suppressor, DAN. DAN is a zinc-finger containing protein that may participate in the cell cycle regulation of DNA synthesis. Both DAN and BAT3 are down-regulated in transformed cells. The interaction with MAPKAP-K3 suggests function either upstream or downstream of this kinase in the inflammatory response.

Another p38-activated protein kinase, MSK-1, was used in a two-hybrid assay and it was found to bind to two proteins. The first, ABLIM, possesses two apparent functional domains: an actin-binding region, and a LIM domain region that is likely involved in protein-protein interactions (Roof et al., 1997). ABLIM may function by coupling the actin-based cytoskeleton to intracellular signaling pathways via its association with MSK-1. This type of function is critical for cell differentiation and morphogenesis, events that occur in response to exposure to external stimuli. This interaction is reminiscent of the interaction between p38 alpha and the cell adhesion/cytoskeleton related protein vinculin, suggesting that phosphorylation of cytoskeletal components may be an important response to inflammatory stimuli.

MSK-1 has also been demonstrated to interact with KIAA0144, a protein of unknown function. The only discernible structural features of KIAA0144 are Ser-, Pro-, and Thr-rich regions.

Analysis of homologous ESTs suggests expression in a large variety of tissues. Interaction with MSK-1 suggests function either as a regulator or a substrate of this kinase.

In a two-hybrid search using the p38-regulated protein kinase PRAK, eleven proteins were identified as PRAK interactors and are therefore implicated in the regulation of inflammatory responses and associated diseases. Two of these proteins, ERK3 and the cAMP-dependent protein kinase (PKA) regulatory subunit, are involved in signal transduction and have been described above as interactors of MAPKAP-K2 in the two-hybrid system. The interaction of ERK3 and PKA with both MAPKAP-K2 and PRAK strengthens the hypothesized role of PKA and ERK3 in the signal transduction cascades that result from inflammatory stimuli.

PRAK interacts with two proteins thought to be involved in vesicular transport. The first protein, Hook1, was isolated based on sequence similarity to the Drosophila Hook protein. The Drosophila homolog is a cytoplasmic coiled-coil protein that functions in the endocytosis of transmembrane receptors and their ligands from the cell surface to the inside of the cell (Kramer et al., 1996). Human Hook1 may participate in signal transduction by internalizing receptors or ligands involved intercellular communication. The second PRAK interactor involved in intracellular protein transport is golgin-95. Golgin-95 is a coiled-coil protein that localizes to the Golgi apparatus (Fritzler et al., 1993; Barr, 1999). Its precise function is unknown, but interestingly, it has been shown to cross-react with certain human autoimmune sera. The interaction of Hook1 and golgin-95 with PRAK suggests these proteins may be substrates of PRAK protein kinase activity, and that PRAK may cause changes in intracellular transport in response to external signals by modulating the activity of these proteins.

PRAK also binds proteins that function in transcriptional regulation, immune response and mitosis. PRAK has been demonstrated to interact with the Prox1 transcription factor. Prox1 is a homeobox-containing protein that has been well studied in mice, and it has been shown to be necessary for the development of the mouse lymphatic system (Wigle et al., 1999). PRAK may be capable of phosphorylating Prox1, thereby affecting its transcriptional function. PRAK has been demonstrated to bind to the immunoglobulin gamma heavy chain constant region. Immunoglobulin molecules recognize antigens and are the first step of the immune response. Although immunoglobulin molecules normally reside outside of the cell, it is possible that PRAK or some other related protein kinase could phosphorylate them to affect their function. This interaction may serve as a direct tie between PRAK and the immune response. PRAK has been shown to interact with kendrin, a large centrosomal protein also called pericentrin. Kendrin forms a complex with gamma tubulin and the dynein motor, and likely plays a critical role in the organization of the mitotic spindle (Purohit et al., 1999). PRAK binding to kendrin suggests that kendrin is a substrate of PRAK; thus, PRAK may play an important function the control of chromosome segregation at mitosis. This interaction is reminiscent of the interaction described above between p38 alpha and the centrosomal protein C-NAP 1, and may serve similar functions.

PRAK has been shown to bind to four proteins for which functions have not yet been determined. The first of these, KIAA0555, was isolated from brain, but analysis of homologous ESTs suggests it is expressed in a variety of tissues. KIAA0555 contains numerous predicted coiled-coil motifs, likely involved in protein-protein interactions, and it displays weak homology (˜20% amino acid identity) to myosin heavy chains from a variety of organisms. We have subsequently identified an interaction between KIAA0555 and protein 14-3-3 epsilon, a member of a large family of proteins involved in signal transduction; the domains with which PRAK and 14-3-3 epsilon interact overlap, suggesting that KIAA0555 may serve as a bridge between PRAK and 14-3-3-dependent signaling pathways. The next PRAK interactor without known function is the leucine-rich protein L130. L130 was described above as an interactor of MAPKAP-K2. Both PRAK and MAPKAP-K2 interact with the same region of L130, arguing that L130 plays in important role in the inflammatory response. The final two PRAK interactors are referred to by their Genbank accession numbers, AL117237 and AL117538. AL117237 was isolated from adult uterus, and analysis of homologous ESTs suggests nearly ubiquitous expression. Analysis of the predicted protein sequence indicates the presence of a coiled-coil region, Arg- and Glu-rich regions, and several nuclear localization signals. AL117538 was isolated from adult testis, and analysis of homologous ESTs suggests expression in a variety of tissues. The predicted protein contains a spectrin repeat and a coiled-coil region. The interaction of these two proteins with PRAK suggests that they may function either as substrates or regulators of the PRAK protein kinase activity and link these two proteins to the inflammatory response and to inflammation-associated diseases.

The proteins disclosed in the present invention were found to interact with their corresponding proteins in the yeast two-hybrid system. Because of the involvement of the corresponding proteins in the physiological pathways disclosed herein, the proteins disclosed herein also participate in the same physiological pathways. Therefore, the present invention provides a list of uses of these proteins and DNA encoding these proteins for the development of diagnostic and therapeutic tools useful in the physiological pathways. This list includes, but is not limited to, the following examples.

Two-Hybrid System

The principles and methods of the yeast two-hybrid system have been described in detail elsewhere (e.g., Bartel and Fields, 1997; Bartel et al., 1993; Fields and Song, 1989; Chevray and Nathans, 1992). The following is a description of the use of this system to identify proteins that interact with a protein of interest.

The target protein is expressed in yeast as a fusion to the DNA-binding domain of the yeast Gal4p. DNA encoding the target protein or a fragment of this protein is amplified from cDNA by PCR or prepared from an available clone. The resulting DNA fragment is cloned by ligation or recombination into a DNA-binding domain vector (e.g., pGBT9, pGBT.C, pAS2-1) such that an in-frame fusion between the Gal4p and target protein sequences is created.

The target gene construct is introduced, by transformation, into a haploid yeast strain. A library of activation domain fusions (i.e., adult brain cDNA cloned into an activation domain vector) is introduced by transformation into a haploid yeast strain of the opposite mating type. The yeast strain that carries the activation domain constructs contains one or more Gal4p-responsive reporter gene(s), whose expression can be monitored. Examples of some yeast reporter strains include Y190, PJ69, and CBY14a. An aliquot of yeast carrying the target gene construct is combined with an aliquot of yeast carrying the activation domain library. The two yeast strains mate to form diploid yeast and are plated on media that selects for expression of one or more Gal4p-responsive reporter genes. Colonies that arise after incubation are selected for further characterization.

The activation domain plasmid is isolated from each colony obtained in the two-hybrid search. The sequence of the insert in this construct is obtained by the dideoxy nucleotide chain termination method. Sequence information is used to identify the gene/protein encoded by the activation domain insert via analysis of the public nucleotide and protein databases. Interaction of the activation domain fusion with the target protein is confirmed by testing for the specificity of the interaction. The activation domain construct is co-transformed into a yeast reporter strain with either the original target protein construct or a variety of other DNA-binding domain constructs. Expression of the reporter genes in the presence of the target protein but not with other test proteins indicates that the interaction is genuine.

In addition to the yeast two-hybrid system, other genetic methodologies are available for the discovery or detection of protein-protein interactions. For example, a mammalian two-hybrid system is available commercially (Clontech, Inc.) that operates on the same principle as the yeast two-hybrid system. Instead of transforming a yeast reporter strain, plasmids encoding DNA-binding and activation domain fusions are transfected along with an appropriate reporter gene (e.g., lacZ) into a mammalian tissue culture cell line. Because transcription factors such as the Saccharomyces cerevisiae Gal4p are functional in a variety of different eukaryotic cell types, it would be expected that a two-hybrid assay could be performed in virtually any cell line of eukaryotic origin (e.g., insect cells (SF9), fingal cells, worm cells, etc.). Other genetic systems for the detection of protein-protein interactions include the so-called SOS recruitment system (Aronheim et al., 1997).

Protein-protein Interactions

Protein interactions are detected in various systems including the yeast two-hybrid system, affinity chromatography, co-immunoprecipitation, subcellular fractionation and isolation of large molecular complexes. Each of these methods is well characterized and can be readily performed by one skilled in the art. See, e.g., U.S. Pat. Nos. 5,622,852 and 5,773,218, and PCT published applications No. WO 97/27296 and WO 99/65939, each of which are incorporated herein by reference.

The protein of interest can be produced in eukaryotic or prokaryotic systems. A cDNA encoding the desired protein is introduced in an appropriate expression vector and transfected in a host cell (which could be bacteria, yeast cells, insect cells, or mammalian cells). Purification of the expressed protein is achieved by conventional biochemical and immunochemical methods well known to those skilled in the art. The purified protein is then used for affinity chromatography studies: it is immobilized on a matrix and loaded on a column. Extracts from cultured cells or homogenized tissue samples are then loaded on the column in appropriate buffer, and non-binding proteins are eluted. After extensive washing, binding proteins or protein complexes are eluted using various methods such as a gradient of pH or a gradient of salt concentration. Eluted proteins can then be separated by two-dimensional gel electrophoresis, eluted from the gel, and identified by micro-sequencing. The purified proteins can also be used for affinity chromatography to purify interacting proteins disclosed herein. All of these methods are well known to those skilled in the art.

Similarly, both proteins of the complex of interest (or interacting domains thereof) can be produced in eukaryotic or prokaryotic systems. The proteins (or interacting domains) can be under control of separate promoters or can be produced as a fusion protein. The fusion protein may include a peptide linker between the proteins (or interacting domains) which, in one embodiment, serves to promote the interaction of the proteins (or interacting domains). All of these methods are also well known to those skilled in the art.

Purified proteins of interest, individually or a complex, can also be used to generate antibodies in rabbit, mouse, rat, chicken, goat, sheep, pig, guinea pig, bovine, and horse. The methods used for antibody generation and characterization are well known to those skilled in the art. Monoclonal antibodies are also generated by conventional techniques. Single chain antibodies are further produced by conventional techniques.

DNA molecules encoding proteins of interest can be inserted in the appropriate expression vector and used for transfection of eukaryotic cells such as bacteria, yeast, insect cells, or mammalian cells, following methods well known to those skilled in the art. Transfected cells expressing both proteins of interest are then lysed in appropriate conditions, one of the two proteins is immunoprecipitated using a specific antibody, and analyzed by polyacrylamide gel electrophoresis. The presence of the binding protein (co-immunoprecipitated) is detected by immunoblotting using an antibody directed against the other protein. Co-immunoprecipitation is a method well known to those skilled in the art.

Transfected eukaryotic cells or biological tissue samples can be homogenized and fractionated in appropriate conditions that will separate the different cellular components. Typically, cell lysates are run on sucrose gradients, or other materials that will separate cellular components based on size and density. Subcellular fractions are analyzed for the presence of proteins of interest with appropriate antibodies, using immunoblotting or immunoprecipitation methods. These methods are all well known to those skilled in the art.

Disruption of Protein-protein Interactions

It is conceivable that agents that disrupt protein-protein interactions can be beneficial in many physiological disorders, including, but not-limited to NIDDM, AD and others disclosed herein. Each of the methods described above for the detection of a positive protein-protein interaction can also be used to identify drugs that will disrupt said interaction. As an example, cells transfected with DNAs coding for proteins of interest can be treated with various drugs, and co-immunoprecipitations can be performed. Alternatively, a derivative of the yeast two-hybrid system, called the reverse yeast two-hybrid system (Leanna and Hannink, 1996), can be used, provided that the two proteins interact in the straight yeast two-hybrid system.

Modulation of Protein-protein Interactions

Since the interaction described herein is involved in a physiological pathway, the identification of agents which are capable of modulating the interaction will provide agents which can be used to track the physiological disorder or to use as lead compounds for development of therapeutic agents. An agent may modulate expression of the genes of interacting proteins, thus affecting interaction of the proteins. Alternatively, the agent may modulate the interaction of the proteins. The agent may modulate the interaction of wild-type with wild-type proteins, wild-type with mutant proteins, or mutant with mutant proteins. Agents can be tested using transfected host cells, cell lines, cell models or animals, such as described herein, by techniques well known to those of ordinary skill in the art, such as disclosed in U.S. Pat. Nos. 5,622,852 and 5,773,218, and PCT published applications No. WO 97/27296 and WO 99/65939, each of which are incorporated herein by reference. The modulating effect of the agent can be screened in vivo or in vitro. Exemplary of a method to screen agents is to measure the effect that the agent has on the formation of the protein complex.

Mutation Screening

The proteins disclosed in the present invention interact with one or more proteins known to be involved in a physiological pathway, such as in NIDDM, AD or pathways described herein. Mutations in interacting proteins could also be involved in the development of the physiological disorder, such as NIDDM, AD or disorders described herein, for example, through a modification of protein-protein interaction, or a modification of enzymatic activity, modification of receptor activity, or through an unknown mechanism. Therefore, mutations can be found by sequencing the genes for the proteins of interest in patients having the physiological disorder, such as insulin, and non-affected controls. A mutation in these genes, especially in that portion of the gene involved in protein interactions in the physiological pathway, can be used as a diagnostic tool and the mechanistic understanding the mutation provides can help develop a therapeutic tool.

Screening for At-risk Individuals

Individuals can be screened to identify those at risk by screening for mutations in the protein disclosed herein and identified as described above. Alternatively, individuals can be screened by analyzing the ability of the proteins of said individual disclosed herein to form natural complexes. Further, individuals can be screened by analyzing the levels of the complexes or individual proteins of the complexes or the mRNA encoding the protein members of the complexes. Techniques to detect the formation of complexes, including those described above, are known to those skilled in the art. Techniques and methods to detect mutations are well known to those skilled in the art.

Techniques to detect the level of the complexes, proteins or mRNA are well known to those skilled in the art.

Cellular Models of Physiological Disorders

A number of cellular models of many physiological disorders or diseases have been generated. The presence and the use of these models are familiar to those skilled in the art. As an example, primary cell cultures or established cell lines can be transfected with expression vectors encoding the proteins of interest, either wild-type proteins or mutant proteins. The effect of the proteins disclosed herein on parameters relevant to their particular physiological disorder or disease can be readily measured. Furthermore, these cellular systems can be used to screen drugs that will influence those parameters, and thus be potential therapeutic tools for the particular physiological disorder or disease. Alternatively, instead of transfecting the DNA encoding the protein of interest, the purified protein of interest can be added to the culture medium of the cells under examination, and the relevant parameters measured.

Animal Models

The DNA encoding the protein of interest can be used to create animals that overexpress said protein, with wild-type or mutant sequences (such animals are referred to as “transgenic”), or animals which do not express the native gene but express the gene of a second animal (referred to as “transplacement”), or animals that do not express said protein (referred to as “knock-out”). The knock-out animal may be an animal in which the gene is knocked out at a determined time. The generation of transgenic, transplacement and knock-out animals (normal and conditioned) uses methods well known to those skilled in the art.

In these animals, parameters relevant to the particular physiological disorder can be measured. These parametes may include receptor function, protein secretion in vivo or in vitro, survival rate of cultured cells, concentration of particular protein in tissue homogenates, signal transduction, behavioral analysis, protein synthesis, cell cycle regulation, transport of compounds across cell or nuclear membranes, enzyme activity, oxidative stress, production of pathological products, and the like. The measurements of biochemical and pathological parameters, and of behavioral parameters, where appropriate, are performed using methods well known to those skilled in the art. These transgenic, transplacement and knock-out animals can also be used to screen drugs that may influence the biochemical, pathological, and behavioral parameters relevant to the Be particular physiological disorder being studied. Cell lines can also be derived from these animals for use as cellular models of the physiological disorder, or in drug screening.

Rational Drug Design

The goal of rational drug design is to produce structural analogs of biologically active polypeptides of interest or of small molecules with which they interact (e.g., agonists, antagonists, inhibitors) in order to fashion drugs which are, for example, more active or stable forms of the polypeptide, or which, e.g., enhance or interfere with the function of a polypeptide in vivo. Several approaches for use in rational drug design include analysis of three-dimensional structure, alanine scans, molecular modeling and use of anti-id antibodies. These techniques are well known to those skilled in the art.

Following identification of a substance which modulates or affects polypeptide activity, the substance may be further investigated. Furthermore, it may be manufactured and/or used in preparation, i.e., manufacture or formulation, or a composition such as a medicament, pharmaceutical composition or drug. These may be administered to individuals.

A substance identified as a modulator of polypeptide function may be peptide or non-peptide in nature. Non-peptide “small molecules” are often preferred for many in vivo pharmaceutical uses. Accordingly, a mimetic or mimic of the substance (particularly if a peptide) may be designed for pharmaceutical use.

The designing of mimetics to a known pharmaceutically active compound is a known approach to the development of pharmaceuticals based on a “lead” compound. This approach might be desirable where the active compound is difficult or expensive to synthesize or where it is unsuitable for a particular method of administration, e.g., pure peptides are unsuitable active agents for oral compositions as they tend to be quickly degraded by proteases in the alimentary canal.

Mimetic design, synthesis and testing are generally used to avoid randomly screening large numbers of molecules for a target property.

Once the pharmacophore has been found, its structure is modeled according to its physical properties, e.g., stereochemistry, bonding, size and/or charge, using data from a range of sources, e.g., spectroscopic techniques, x-ray diffraction data and NMR. Computational analysis, similarity mapping (which models the charge and/or volume of a pharmacophore, rather than the bonding between atoms) and other techniques can be used in this modeling process.

A template molecule is then selected, onto which chemical groups that mimic the pharmacophore can be grafted. The template molecule and the chemical groups grafted thereon can be conveniently selected so that the mimetic is easy to synthesize, is likely to be pharmacologically acceptable, and does not degrade in vivo, while retaining the biological activity of the lead compound. Alternatively, where the mimetic is peptide-based, further stability can be achieved by cyclizing the peptide, increasing its rigidity. The mimetic or mimetics found by this approach can then be screened to see whether they have the target property, or to what extent it is exhibited. Further optimization or modification can then be carried out to arrive at one or more final mimetics for in vivo or clinical testing.

Diagnostic Assays

The identification of the interactions disclosed herein enables the development of diagnostic assays and kits, which can be used to determine a predisposition to or the existence of a physiological disorder. In one aspect, one of the proteins of the interaction is used to detect the presence of a “normal” second protein (i.e., normal with respect to its ability to interact with the first protein) in a cell extract or a biological fluid, and further, if desired, to detect the quantitative level of the second protein in the extract or biological fluid. The absence of the “normal” second protein would be indicative of a predisposition or existence of the physiological disorder. In a second aspect, an antibody against the protein complex is used to detect the presence and/or quantitative level of the protein complex. The absence of the protein complex would be indicative of a predisposition or existence of the physiological disorder.

Nucleic Acids and Proteins

A nucleic acid or fragment thereof has substantial identity with another if, when optimally aligned (with appropriate nucleotide insertions or deletions) with the other nucleic acid (or its complementary strand), there is nucleotide sequence identity in at least about 60% of the nucleotide bases, usually at least about 70%, more usually at least about 80%, preferably at least about 90%, and more preferably at least about 95-98% of the nucleotide bases. A protein or fragment thereof has substantial identity with another if, optimally aligned, there is an amino acid sequence identity of at least about 30% identity with an entire naturally-occurring protein or a portion thereof, usually at least about 70% identity, more usually at least about 80% identity, preferably at least about 90% identity, and more preferably at least about 95% identity.

Identity means the degree of sequence relatedness between two polypeptide or two polynucleotides sequences as determined by the identity of the match between two strings of such sequences, such as the full and complete sequence. Identity can be readily calculated. While there exist a number of methods to measure identity between two polynucleotide or polypeptide sequences, the term “identity” is well known to skilled artisans (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). Methods commonly employed to determine identity between two sequences include, but are not limited to those disclosed in Guide to Huge Computers, Martin J. Bishop, ed., Academic Press, San Diego, 1994, and Carillo, H., and Lipman, D., SIAM J Applied Math. 48: 1073 (1988). Preferred methods to determine identity are designed to give the largest match between the two sequences tested. Such methods are codified in computer programs. Preferred computer program methods to determine identity between two sequences include, but are not limited to, GCG (Genetics Computer Group, Madison Wis.) program package (Devereux, J., et al., Nucleic Acids Research 12(1). 387 (1984)), BLASTP, BLASTN, FASTA (Altschul et al. (1990); Altschul et al. (1997)). The well-known Smith Waterman algorithm may also be used to determine identity.

Alternatively, substantial homology or similarity exists when a nucleic acid or fragment thereof will hybridize to another nucleic acid (or a complementary strand thereof) under selective hybridization conditions, to a strand, or to its complement. Selectivity of hybridization exists when hybridization which is substantially more selective than total lack of specificity occurs. Nucleic acid hybridization will be affected by such conditions as salt concentration, temperature, or organic solvents, in addition to the base composition, length of the complementary strands, and the number of nucleotide base mismatches between the hybridizing nucleic acids, as will be readily appreciated by those skilled in the art. Stringent temperature conditions will generally include temperatures in excess of 30° C., typically in excess of 37° C., and preferably in excess of 45° C. Stringent salt conditions will ordinarily be less than 1000 mM, typically less than 500 mM, and preferably less than 200 mM. However, the combination of parameters is much more important than the measure of any single parameter. See, e.g., Asubel, 1992; Wetmur and Davidson, 1968.

The terms “isolated”, “substantially pure”, and “substantially homogeneous” are used interchangeably to describe a protein or polypeptide which has been separated from components which accompany it in its natural state. A monomeric protein is substantially pure when at least about 60 to 75% of a sample exhibits a single polypeptide sequence. A substantially pure protein will typically comprise about 60 to 90% W/W of a protein sample, more usually about 95%, and preferably will be over about 99% pure. Protein purity or homogeneity may be indicated by a number of means well known in the art, such as polyacrylamide gel electrophoresis of a protein sample, followed by visualizing a single polypeptide band upon staining the gel. For certain purposes, higher resolution may be provided by using HPLC or other means well known in the art which are utilized for purification.

Large amounts of the nucleic acids of the present invention may be produced by (a) replication in a suitable host or transgenic animals or (b) chemical synthesis using techniques well known in the art. Constructs prepared for introduction into a prokaryotic or eukaryotic host may comprise a replication system recognized by the host, including the intended polynucleotide fragment encoding the desired polypeptide, and will preferably also include transcription and translational initiation regulatory sequences operably linked to the polypeptide encoding segment. Expression vectors may include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, and mRNA stabilizing sequences. Secretion signals may also be included where appropriate which allow the protein to cross and/or lodge in cell membranes, and thus attain its functional topology, or be secreted from the cell. Such vectors may be prepared by means of standard recombinant techniques well known in the.

### EXAMPLES

The present invention is further detailed in the following Examples, which are offered by way of illustration and are not intended to limit the invention in any manner. Standard techniques well known in the art or the techniques specifically described below are utilized.

### Example 1

### Yeast Two-Hybrid System

The principles and methods of the yeast two-hybrid systems have been described in detail (Bartel and Fields, 1997). The following is thus a description of the particular procedure that we used, which was applied to all proteins.

The cDNA encoding the bait protein was generated by PCR from brain cDNA. Gene-specific primers were synthesized with appropriate tails added at their 5′ ends to allow recombination into the vector PGBTQ. The tail for the forward primer was 5′-GCAGGAAACAGCTATGACCATACAGTCAGCGGCCGCCAcC-3¹ (SEQ ID NO:1) and the tail for the reverse primer was 5′-ACGGCCAGTCGCGTGGAGTGTTATGTCATGCGGCCGCTA-3′ (SEQ ID NO:2). The tailed PCR product was then introduced by recombination into the yeast expression vector pGBTQ, which is a close derivative of pGBTC (Bartel et al., 1996) in which the polylinker site has been modified to include M13 sequencing sites. The new construct was selected directly in the yeast J693 for its ability to drive tryptophane synthesis (genotype of this strain: Mat α, ade2, his3, leu2, trpl, URA3::GAL1-lacZ LYS2::GAL1-HIS3 gal4del gal80del cyhR2). In these yeast cells, the bait is produced as a C-terminal fusion protein with the DNA binding domain of the transcription factor Gal4 (amino acids 1 to 147). A total human brain (37 year-old male Caucasian) cDNA library cloned into the yeast expression vector pACT2 was purchased from Clontech (human brain MATCHMAKER cDNA, cat. # HL4004AH), transformed into the yeast strain J692 (genotype of this strain: Mat a, ade2, his3, leu2, trpl, URA3::GAL1-lacZ LYS2::GAL1-HIS3 gal4del gal80del cyhR2), and selected for the ability to drive leucine synthesis. In these yeast cells, each cDNA is expressed as a fusion protein with the transcription activation domain of the transcription factor Gal4 (amino acids 768 to 881) and a 9 amino acid hemagglutinin epitope tag. J693 cells (Mat a type) expressing the bait were then mated with J692 cells (Mat a type) expressing proteins from the brain library. The resulting diploid yeast cells expressing proteins interacting with the bait protein were selected for the ability to synthesize tryptophan, leucine, histidine, and β-galactosidase. DNA was prepared from each clone, transformed by electroporation into E. coli strain KC8 (Clontech KC8 electrocompetent cells, cat. # C2023-1), and the cells were selected on ampicillin-containing plates in the absence of either tryptophane (selection for the bait plasmid) or leucine (selection for the brain library plasmid). DNA for both plasmids was prepared and sequenced by di-deoxynucleotide chain termination method. The identity of the bait cDNA insert was confirmed and the cDNA insert from the brain library plasmid was identified using BLAST program against public nucleotides and protein databases. Plasmids from the brain library (preys) were then individually transformed into yeast cells together with a plasmid driving the synthesis of lamin fused to the Gal4 DNA binding domain. Clones that gave a positive signal after galactosidase assay were considered false-positives and discarded. Plasmids for the remaining clones were transformed into yeast cells together with plasmid for the original bait. Clones that gave a positive signal after -galactosidase assay were considered true positives.

### Example 2

### Identification of p38 Alpha/CYT4 Interaction

A yeast two-hybrid system as described in Example 1 using amino acids 194-319 of p38 alpha (Swiss Protein (SP) accession no. Q13083) as bait was performed. One clone that was identified by this procedure included amino acids 4-218 of CYT4 (GenBank (GB) accession no. AF075458).

### Example 3

### Identification of MAPKAP-K3/PN2012 Interaction

A yeast two-hybrid system as described in Example 1 using amino acids encoded by nucleotides 92-1003 of MAPKAP-K3 (GB accession no. U9578) as bait was performed. One clone that was identified by this procedure included novel protein PN2012. The DNA sequence and the predicted protein sequence for PN2012 are set forth in Tables 32 and 33, respectively. The start codon and stop codon are bolded in Table 32. Several variants were also found, including:

T is substituted for C at nucleotide position 1190,

C is subsituted for T as nucleotide position 2839,

A is substituted for G at nucleotide position 3338,

G is substitued for A at nucleotide position 4753, and

nucleotides at positions 723-725 are deleted (also underlined in Table 32).

TABLE 32
Nucleotide Sequence ot PN2012

gccgcgtcgacgtcgacccagactggagcgacgtttaaagaaggggcagaatcgctggggagtgcggcttcttcttgttgggggactcccagc
cttccgcgcgtccggaggaggagaagcggcggcgccgggaagcaggcatggagagtagaaaactgatttctgctacagacattcagtactct
ggcagtctgctgaactccttgaatgagcaacgtggccatggactcttctgtgatgttaccgttattgtggaagaccgaaaattccgggctcacaaga
tggtgtcccattgtcacaggttaaaagcatctcaggtacagcgcaggatggtaatactgagcctttacctcctgattctggtgacaagaaccttgtaa
tacagaaatcaaaagatgaagcccaagataatggggctactataatgcctattataacagagtctttttcattatctgccgaagattatgaaatgaaaa
agatcattgttaccgattctgatgatgatgatgatgatgatgtcattttttgctccgagattctgcccacaaaggagactttgccgagtaataacacagt
ggcacaggtccaatctaacccaggccctgttgctatttcagatgttgcacctagtgctagcaataactcgccccctttaacaaatatcacacctactc
agaaacttcctactcctgtgaatcaggcaactttgagccaaacacaaggaagtgaaaaattgttggtatcttcagctccaacacatctgactcccaat
agacaccaaacagtgctattttaacaggaaacaaggccaatgaagaggaggaggaggaaataatagatgatgatgatgacactattagctccag
tcctgactcggccgtcagtaatacatctttggtcccacaggctgatacctcccaaaataccagttttgatggatcattaatacagaagatgcagattcc
ggtcagaagatcattactttagatacagctactgaaattgaaggcttatcgactggttgcaaggtttatgcaaatatcggtgaagatacttatgatata
gtgatccctgtcaaagatgaccctgatgaaggggaggccagacttgagaatgaaataccaaaaacgtctggcagcgagatggcaaacaaacgt
tgcggagacattttaacattcattcttgggagaagaagtatccgtgccgttactgtgagaaggtatttcctcttgcagaatatcgcacaaagcatgaa
attcatcacacaggggagcgaaggtatcagtgtttggcctgtggcaaatctttcatcaactatcagtttatgtcttcacatataaagtcagttcatagtc
gcaatgaaggatgatggtattgggtataaggttgacactggaaaagaacctccagtagggaccactacatctactcagaacaagccaatgacctg
ggaagatatttttattcagcaggaaaatgattcaatttttaaacaaaatgtaacagatggcagtactgagtttgaatttataataccagagtcttactaa
actcctttgaaatactagaaagttttgttttggatgatggggcaggggtttcagaagatctgtaaaacaaattaaggtgcgaacaagttaatttgatct
gccacattatctgaaggaagtgtagtgggatttttgttgataatttttagaagcaaattttcctgaaagttttgagtagaggtgagaccccctccccaag
tatctgtttatatagttagttttcagctcatttaaaagaggcaaaaattaaaagcttggagagatagtttcctgaatagaatttgaagcagtctgaatgttc
tgctagtccaagcgaacagcaacctcctgctaccctccctctatgaaaatagccatgcagacaagtctctcatctgaagaacaaattagatttagcta
aatgaaaacaatctctgtaatggcagataggaggagatgaaaagttctgttgcatggatttttaattctctggctaccacatagtagagaatggaatg
cacatatttaaacacttagaaaataaagttaacacttactgaagtgctagtactaaactgtgctagtactaaaagaaaacaggttggaacatacatata
cgatgtgaatgatacggatgccggcagagcttccagatctttcagactcaactgctaggtcaattagtttgtcataataaaacttggcagattctacaa
tgaatatgacatttaaactctgtgcctactaaaggtatcttctggagtttttgggaggagagaaactggaaaattaaattgtatttttgccagaagactct
cacacacaaccactatgaagaaataatgccgcatttttcccccattgtaccaaaaagataaaaaaatggtaaacactgatcaaggtattttgtattgtc
atttaattctgaaaaaaaaaaaaaaaaaaaaaaaaaaaa (SEQ ID NO:3)

TABLE 33
Predicted Amino Acid Sequence of PN2012

MESRKLISATDIQYSGSLLNSLNEQRGHGLFCDVTVIVEDRKFRAHKNILSASSTYFHQLFSV
AGQVVELSFIRAEIFAEILNYIYSSKIVRVRSDLLDELIKSGQLLGVKFIAELGVPLSQVKSISG
TAQDGNTEPLPPDSGDKNLVIQKSKDEAQDNGATIMPIITESFSLSAEDYEMKKIVTDSDDD
DDDDVIFCSEILPTKETLPSNNTVAQVQSNPGPVAJSDVAPSASNNSPPLTMTPTQKIPTPVN
QATLSQTQGSEKLLVSSAPTHLTPNJILLNQTPLSTPPNVSSSLPNIIMPSSTNLLVQNQQTPNS
AILTGKKANEEEEEEIIDDDDDTISSSPDSAVSNTSLMPQADTSQNTSFDGSLIQKMQJPTLLQ
EPLSNSLKTSDIITRNTNDPGVGSKHLMEGQKIITLDTATEIEGLSTGCKVYAMGEDTYDIVTP
VKDDPDEGEARLENEIPKTSGSEMANKRMKVKHDDHYELIVDGRVYYICIVCKRSYVCLTS
LRRHFNIHSWEKKYPCRYCEKVFPLAEYRTKHEIHHTGERRYQCLACGKSFINYQFMSSHIK
SVHSQDPSGDSKLYRLHPCRSLQIRQYAYLSDRSSTIPAMIKDDGIGYKVDTGKFPPVGTTTS
TQNKIPMTWEDTFIQQIENDSIFKQNVTDGSTEFEFIIPESY (SEQ ID NO:4)

### Example 4

### Identification of MAPKAP-K3/PN7771 Interaction

A yeast two-hybrid system as described in Example 1 using amino acids encoded by nucleotides433-1003 of MAPKAP-K3 (GB accession no. U9578) as bait was performed. One clone that was identified by this procedure included novel protein fragment PN7771. The DNA sequence and the predicted protein sequence for PN7771 are set forth in Tables 34 and 35, respectively.

TABLE 34
Nucleotide Sequence of PN7771

cttattttgaaaacatttacatagtgattagttaacccaacagaccaatcctgggaagacagccagagcctgcagcaccttagtaacagaaaaactg
ataattaggagaagagacctgtccaagaccaggaacctggaccaaaattgtgccatgttgctttactttaatgagtggccccagtaaaaactgagct
ctgctttttttatagtcaaaggactggttctgagagccttgttgcagatggctgaggtcaccgtcccaagggtgtatgtcgtgtttggcatccattgcat
catggcgaaggcatcttcagatgtgcaggtttcaggctttcatcggaaaatccagcacgttaaaaatgaactttgccacatgttgagcttggaggag
gtggccccagtgctgcagcagacattacttcaggacaacctcttgggcagggtacattttgaccaatttaaagaagcattaatactcatcttgtccag
aactctgtcaaatgaagaacactttcaagaaccagactgctcactagaagctcagcccaaatatgttagaggtgggaagcgttacggacgaaggt
ccttgcccgagttccaagagtccgtggaggagtttcctgaagtgacggtgattgagccactggatgaagaagcgcggccttcacacatcccagc
cggtgactgcagtgagcactggaagacgcaacgcagtgaggagtatgaagcggaaggccagttaaggttttggaacccagatgacttgaatgc
ttcacagagtggatcttcccctccocaagactggatagaagagaaactgcaagaagtttgtgaagatttggggatcacccgtgatggtcacctgaa
ccggaagaagctggtctccatctgtgagcagtatggtttacagaatgtggatggagagatgctcgaggaagtattccataatcttgatcctgacggt
catgcagtctttcgatgagagtggacgacgtaccacaacctcatcagcaatgacaagtaccattggctttcgggtcttctcctgcctggatgatggg
atgggccatgcatctgtggagagaatactggacacctggcaggaagagggcattgagaacagccaggagatcctgaaggcottggatttcagc
ctcgatggaaacatcaatttgacagaattaacactggcccttgaaaatgaacttttggttaccaagaacagcattcaccaggcggctctggccagct
ttaaggctgaaatccggcatttgttggaacgagttgatcaggtggtcagagaaaaagagaagctacggtcagatctggacaaggccgagaagct
caagtctttaatggcctcggaggtggatgatcaccatgcggccatagagcggcggaatgagtacaacctcaggaaactggatggagagtacaa
ggagcgaatagcagccttaaaaaatgaactccgaaaagagagagagcagatcctgcagcaggcaggcaagcagcgtttagaacttgaacagg
aaattgaaaaggcaaaaacagaagagaactatatccgggaccgccttgccctctctttaaaggaaaacagtcgtctggaaaatgagcttctagaa
aatgcagagaagttggcagaatatgagaatctgacaaacaaacttcagagaaatttggaaaatgtgttagcagaaaagtttggtgacctcgatcct
agcagtgctgagttcttcctgcaagaagagagactgacacagatgagaaatgaatatgagcggcagtgcagggtactacaagaccaagtagatg
aactccagtctgagctggaagaatatcgtgcacaaggcagagtgctcaggcttccgttgaagaactcaccgtcagaagaagttgaggctaacag
cggtggcattgagcccgaacacgggctcggttctgaagaatgcaatccattgaatatgagcattgaggcagagctggtcattgaacagatgaaag
aacaacatcacagggacatatgttgcctcagactggagctcgaagataaagtgcgccattatgaaaagcagctggacgaaaccgtggtcagctg
caagaaggcacaggagaacatgaagcaaaggcatgagaacgaaacgcgcaccttagaaaaacaaataagtgaccttaaaaatgaaattgctga
acttcaggggcaagcagcagtgctcaaggaggcacatcatgaggccacttgcaggcatgaggaggagaaaaaacaactgcaagtgaagcttg
aggaggaaaagactcacctgcaggagaagctgaggctgcaacatgagatggagctcaaggctagactgacacaggctcaagcaagctttgag
cgggagagggaaggccttcagagtagcgcctggacagaagagaaggtgagaggcttgactcaggaactagagcagtttcaccaggagcagc
tgacaagcctggtggagaaacacactcttgagaaagaggagttaagaaaagagctcttggaaaagcaccaaagggagcttcaggagggaagg
gaaaaaatggaaacagagtgtaatagaagaacctctcaaatagaagcccagtttcagtctgattgtcagaaagtcactgagaggtgtgaaagcgc
tctgcaaagcctggaggggcgctaccgccaagagctgaaggacctccaggaacagcagcgtgaggagaaatcccagtgggaatttgagaag
gacgagctcacccaggagtgtgcggaagcccaggagctgctgaaagagactcttaagagagagaaaacaacttctctggtcctgacccagga
gagagagatgctggagaaaacatacaaagaacatttgaacagcatggtcgtcgagagacagcagctactccaagacctggaagacctaagaaa
tgtatctgaaacccagcaaagcctgctgtctgaccagatacttgagctgaagagcagtcacaaaagggaactgagggagcgtgaggaggtcct
gtgccaggcaggggcttcggagcagctggccagccagcggctggaaagactagaaatggaacatgaccaggaaaggcaggaaatgatgtcc
aagcttctagccatggagaacattcacaaagcgacctgtgagacagcagatcgagaaagagccgagatgagcacagaaatctccagacttcag
agtaaaataaaggaaatgcagcaggcaacatctcctctctcaatgcttcagagtggttgccaggtgataggagaggaggaggtggaaggagatg
gagccctgtccctgcttcagcaaggggagcagctgttggaagaaaatggggacgtcctcttaagcctgcagagagctcatgaacaggcagtga
aggaaaatgtgaaaatggctactgaaatttctagattgcaacagaggctacaaaagttagagccagggttagtaatgtcttcttgtttggatgagcca
gctactgagttttttggaaatactgcggaacaaacagagcagtttttacagcaaaaccgaacgaagcaagtagaaggtgtgaccaggcggcatgt
cctaagtgacctggaagatgatgaggtccgggacctgggaagtacagggacgagctctgttcagagacaggaagtcaaaatagaggagtctga
agcttcagtagagggtttttctgagcttgaaaacagtgaagagaccaggactgaatcctgggagctgaagaatcagattagtcagcttcaggaaca
gctaatgatgttatgtgcggactgtgatcgagcttctgaaaagaaacaggacctactttttgatgtttctgtgctaaaaaagaaactgaagatgcttga
gagaatccctgaggcttctcccaaatataagctgttgtatgaagatgtgagccgagaaaatgactgccttcaggaagagctgagaatgatggaga
cacgctacgatgaggcactagaaaataacaaagaactcactgcagaggttttcaggttgcaggatgagctgaagaaaatggaggaagtcactga
aacattcctcagcctggaaaagagttacgatgaggtcaaaatagaaaatgaggggctgaatgttctggttttgagacttcaaggcaagattgagaa
gcttcaggaaagcgtggtccagcggtgtgactgctgcttatgggaagccagtttagagaacctggaaatcgaacctgatggaaatatactccagc
tcaatcagacactggaagagtgtgtgcccagggttaggagtgtacatcatgtcatagaggaatgtaagcaagaaaaccagtaccttgaggggaa
cacacagctcttggaaaaagtaaaagcacatgaaattgcctggttacatggaacaattcagacacatcaagaaaggccaagagtacagaatcaa
gttatactggaggaaaacactactctcctaggctttcaagacaaacattttcagcatcaggccaccatagcagagttagaactggagaaaacaaag
ttacaggagctgactaggaagttgaaggagagagtcactattttagttaagcaaaaagatgtactttctcacggagaaaaggaggaagagctgaa
ggcaatgatgcatgacttgcagatcacgtgcagtgagatgcagcaaaaagttgaacttctgagatatgaatctgaaaagcttcaacaggaaaattc
tattttgagaaatgaaattactactttaaatgaagaagatagcatttctaacctgaaattagggacattaaatggatctcaggaagaaatgtggcaaaa
aacggaaactgtaaaacaagaaaatgctgcagttcagaagatggttgaaaatttaaagaaacagatttcagaattaaaaatcaaaaaccaacaatt
ggatttggaaaatacagaacttagccaaaagaactctcaaaaccaggaaaaactgcaagaacttaatcaacgtctaacagaaatgctatgccaga
aggaaaaagagccaggaaacagtgcattggaggaacgggaacaagagaagtttaatctgaaagaagaactggaacgttgtaaagtgcagtcct
ccactttagtgtcttctctggaggcggagctctctgaagttaaaatacagacccatattgtgcaacaggaaaaccaccttctcaaagatgaactgga
gaaaatgaaacagctgcacagatgtcccgatctctctgacttccagcaaaaaatctctagtgttctaagctacaacgaaaaactgctgaaagaaaa
ggaagctctgagtgaggaattaaatagctgtgtcgataagttggcaaaatcaagtcttttagagcatagaattgcgacgatgaagcaggaacagaa
atcctgggaacatcagagtgcgagcttaaagtcacagctggtggcttctcaggaaaaggttcagaatttagaagacaccgtgcagaatgtaaacc
tgcaaatgtcccggatgatatctgacctacgagtgactcagcaggaaaaggaggctttaaaacaagaagtgatgtctttacataagcaacttcaga
atgctggtggcaagagctgggccccagagatagctactcatccatcagggctccataaccagcagaaaaggctgtcctgggacaagttggatca
tctgatgaatgaggaacagcagctgctttggcaagagaatgagaggctccagaccatggtacagaacaccaaagccgaactcacgcactcccg
ggagaaggtccgtcaattggaatccaatcttcttcccaagcaccaaaaacatctaaacccatcaggtaccatgaatcccacagagcaagaaaaatt
gagcttaaagagagagtgtgatcagtttcagaaagaacaatctcctgctaacaggaaggtcagtcagatgaattcccttgaacaagaattagaaac
aattcatttggaaaatgaaggcctgaaaaagaaacaagtaaaactggatgagcagctcatggagatgcagcacctgaggtccactgcgacgcct
agcccgtcccctcatgcttgggatttgcagctgctccagcagcaagcctgtccgatggtgcccagggagcagtttctgcagcttcaacgccagct
gctgcaggcagaaaggataaaccagcacctgcaggaggaacttgaaaacaggacctccgaaaccaacacaccacagggaaaccaggaaca
actggtaactgtcatggaggaacgaatgatagaagttgaacagaaactgaaactagtgaaaaggcttcttcaagagaaagtgaatcagctcaaag
aacaactctgcaagaacactaaggcagacgcaatggtgaaggacttgtatgttgaaaatgcccagttgttgaaagctctggaagtgactgaacag
cgacagaaaacagcagagaagaaaaattacctcctggaggagaagattgccagcctcagtaatatagttaggaatctgacaccagcgccattga
taattggttaggaggcagggcttattaagtggttattaaccgctgacatcagacaaacccaaatctgtagaattctaacctcctaacacctgtgacag
aaaagtttcatttggggagctggtcttctaagaaacggataaagccacataattaaagcagttgaactagagggaaagcactgaacaaaccacttt
catgagacaataatccgaaaagttcgctttgatatattcctggagggccaagcccatctatttacaaaaggtgaacagcaaaatcaagcactgcttt
atgggcaggaacacaagagaaagcaaactgcccaagaagtcatcatgtcagaaactcaatctcaacaaaataatttccatcagggaacttcagg
gtttcttgggggcttatgagtctcaccggtcaacccaggaggcctcactacaagagccttgacaaggcactgttttttgtgggactgggagttcaca
gagaaataccatacagataccttatatgtatgcatttgtgcaacaatttttgagaaggtgagtggcaatttataatttagttggcaatttataatagaact
aaagggaatcaagtttgcctttgagataatacgttacactaagaaaaggaaaatgtggatagtaaaacccacctctctcatcctattgtactctcttct
gctttttagaagcctgcacttaagcttagatttgtgaagggagagtagaaggggagaagtagaaccacagtgttttatttatttttctaaaactcttact
attggaatgatataattatacaagtaatgccaaaaaccaagtcaaagcctaattaaccaaagcactcatttaaaaatcatcatgtttggacctatctgg
attttgcaaacatgaaaaaaaaaaaaaaaaa (SEQ ID NO:5)

TABLE 35
Predicted Amino Acid Sequence of PN7771

MAEVTVPRVYVVFGIHCIMAKASSDVQVSGFHRKIQHVKNELCHMLSLEEVAPVLQQTLL
QDNLLGRVHFDQFKEALILILSRTLSNEEHFQEPDCSLEAQPKYVRGGKRYGRRSLPEFQES
VEEFPEVTVIEPLDEEARPSHIPAGDCSEHWKTQRSEEYEAEGQLRFWNPDDLNASQSGSSP
PQDWIEEKLQEVCEDLGITRDGHLNRKKLVSICEQYGLQNVDGEMLEEVFJINLDPDGTMS
VEDFFYGLFKINGKSLTPSASTPYRQLKRIILSMQSFDESGRRTTTSSAMTSTJGFRVFSCLDD
GMGHASVERLLDTWQEEGIENSQEILKALDFSLDGNINLTELTLALENELLVTKNSIIIQAAL
ASFKAEIRHLLERVDQVVREKEKLRSDLDKAEKLKSLMASEVDDHHAAIERRNEYNLRKL
DGEYKERIAALKNELRKEREQJLQQAGKQRLELEQEIEKAKTEENYIRDRLALSLKIENSRLE
NELLENAEKLAEYENLTNKLQRNLENYLAEKFGDLDPSSAEFFLQEERLTQMRNEYERQCR
VLQDQVDELQSELEEYRAQGRVLRLPLKNSPSEEVEANSGGIEPEHGLGSEECNPLNMSIEA
ELVIEQMKEQHHRDICCLRLELEDKVRHYEKQLDETVVSCKKAQENMKQRHENETRTLEK
QISDLKNEIAELQGQAAVLKEAHHEATCRHEEEKKQLQVKJEEEKTHLQEKLRLQHEMEL
KARLTQAQASFEREREGLQSSAWTEEKVRGLTQELEQFHQEQLTSLVEKHTLKEELRKEL
LEKHQRELQEGREKMETECNRRTSQIEAQFQSDCQKVTERCESALQSLEGRYRQELKDLQE
LQSGCQVIGEEEVEGDGALSLLQQGEQLLEENGDVLLSLQRAHEQAVKENVKMATEISRLQ
QRLQKLEPGLVMSSCLDEPATEFFGNTAEQTEQFLQQNRTKQVEGVTRRHVLSDLEDDEVR
DLGSTGTSSVQRQEVKIEESEASVEGFSELENSEETRTESWELKNQISQLQEQLMMLCADCD
RASEKKQDLLFDVSVLKKKLKMLERIPEASPKYKLLYEDVSRENDCLQEELRMMETRYDE
ALENNKELTAEVFRLQDELKKMEEVTETFLSLEKSYDEVKIENEGLNVLVLRLQGKIEKLQE
DLGSTGTSSVQRQEVKIEESEASVEGFSELENSEETRTESWELKNQISQLQEQLMMLCADCD
RASEKKQDLLFDVSVLKKKLKMLERIPEASPKYKLLYEDVSRENDCLQEELRMMETRYDE
ALENNKELTAEVFRLQDELKKMEEVTETFLSLEKSYDEVKIENEGLNVLVLRLQGKIEKLQE
SVVQRCDCCLWEASLENLEIEPDGNILQLNQTLEECVPRVRSVTIIWIEECKQENQYLEGNT
QLLEKVKAHEIAWLHGTJQTHQERPRVQNQVILEENTTLLGFQDJQIFQHQATIAELELEKTK
LQELTRKLKERVTILVKQKDVLSHGEKEEELKAMMHDLQITCSEMQQKVELLRYESEKLQ
QENSILRNEITTNEEDSISNLKLGTLNGSQEEMWQKTETVKQENAAVQKMVENLKKQISE
LKIKNQQLDLENTELSQKNSQNQEKLQELNQRTEMLCQKEKEPGNSALEEREQEKFNLKE
ELERCKVQSSTLVSSLEAELSEVKIQTHIVQQENHLLKDELEKMKQLHRCPDLSDFQQKISS
VLSYNEKLLKEKEALSEELNSCVDKLAKSSLLEHRIATMKQEQKSWEHQSASLKSQLVASQ
EKVQNLEDTVQNVNLQMSRMKSDLRVTQQEKEALKQEVMSLHKQLQNAGGKSWAPEIAT
HPSGLHNQQKRLSWDKLDHLMNEEQQLLWQENERQTMVQNTKAELTHSREKVRQLESN
LLPKHQKHLNPSGTMNPTEQEKLSLKRECDQFQKEQSPANRKVSQMNSLEQELETIHLENE
GLKKKQVKLDEQLMEMQHLRSTATPSPSPHAWDLQLLQQQACPMVPREQFLQLQRQLLQ
AERINQHLQEELENRTSETNTPQGNQEQLVTVMEERMIEVEQKLKLVKRLLQEKVNQLKEQ
LCKNTKADAMVKDLYVENAQLLKALEVTEQRQKTEAKKNYLLEEKIASLSNIVNLTPAPL
TSTPPLRS (SEQ ID NO:6)

### Example 5

### Identification of PRAK/PN7098 Interaction

A yeast two-hybrid system as described in Example 1 using amino acids encoded by nucleotides 786-1104 of PRAK (GB accession no. AF032437) as bait was performed. One clone that was identified by this procedure included novel protein fragment PN7098. The DNA sequence and the predicted protein sequence for PN7098 are set forth in Tables 36 and 37 respectively.

TABLE 36
Nucleotide Sequence of PN7098

gccttggattttcaggttttcatcctgatacttgtttacttttctggggcagaaaagcttgcactaattgctctccatggtggctaatttttcaagagcttg
attttaccttacattcataagctttgcaaaggaatgtttacaaagaaattgggaaatacaaacaaaaacaaagagtatcgtcagcagaaaaaggatc
aagacttccccactgctggccagaccaaatcccccaaattttcttacacttttaaaagcactgtaaagaagattgcaaagtgttcatccactcacaact
tatccactgaggaagacgaggccagtaaagagttttccctctcaccaacattcagttaccgagtagctattgccaatggcctacaaaagaatgctaa
agtaaccaccagtgataatgaggatctgcttcaagagctctcttcaatcgagagttcctactcagaatcattaaatgaactaaggagtagcacagaa
aaccaggcacaatcaacacacacaatgccagttagacgcaacagaaagagttcaagcagccttgcaccctctgagggcagctctgacgggga
gcgtactctacatggcttaaaactgggagctttacgaaaactgagaaaatggaaaaaagagtcaagaatgtgtctcctcagactcagagttaagcac
catgaaaaaatcctggggaataagaagtaagtctttggacagaactgtccgaaacccaaagacaaatgccctggagccagggttcagttcctctg
gctgcattagccaaacacatgatgtcatggaaatgatctttaaggaacttcagggaataagtcagattgaaacagaactttctgaactacgagggca
cgtcaatgctctcaagcactccatcgatgagatctccagcagtgtggaggttgtacaaagtgaaattgagcagttgcgcacagggtttgtccagtct
cggagggaaactagagacatccatgattatattaagcacttaggtcatatgggtagcaaggcaagcctgagatttttaaatgtgactgaagaaagat
ttgaatatgttgaaagcgtggtgtaccaaattctaatagataaaatgggtttttcagatgcaccaaatgctattaaaattgaatttgctcagaggatagg
acaccagagagactgcccaaatgcaaagcctcgacccatacttgtgtactttgaaacccctcaacaaagggattctgtcttaaaaaagtcatataaa
ctcaaaggaacaggcattggaatctcaacagatattctaactcatgacatcagagaaagaaaagagaaagggataccatcctcccagacatatga
gagcatggctataaagttgtctactccagagccaaaaatcaagaagaacaattggcagtcacctgatgacagtgatgaagatcttgaatctgacct
caatagaaacagttacgctgtgctttccaagtcagagcttctaacaaagggaagtacttccaagccaagctcaaaatcacacagtgctagatccaa
gaataaaactgctaatagcagcagaatttcaaataaatcagattatgataaaatctcctcacagttgccagaatcagatatcttggaaaagcaaacca
caacccattatgcagatgcaacacctctctggcactcacagagtgattttttcactgctaaacttagtcgttctgaatcagatttttccaaattgtgtcag
tcttactcagaagatttttcagaaaatcagtttttcactagaactaatggaagctctctcctgtcatcttcggaccgggagctatggcagaggaaacag
gaaggaacagcgaccctgtatgacagtcccaaggaccagcatttgaatggaagtgttcagggtatccaagggcagactgaaactgaaaacaca
gaaactgtggatagtggaatgagtaatggcatggtgtgtgcatctggagaccggagtcattacagtgattctcagctctctttacatgaggatctttct
ccatggaaggatggaatcaaggagctgatttaggcttggattcatccacccaggaaggttttgattatgaaacaaacagtctttttgaccaacagct
tgatgtttacaataaagacctagaatacttgggaaagtgccacagtgatcttcaagatgactcagagagctacgacttaactcaagatgacaattctt
ctccatgccctggcttggataatgaaccacaaggccagtgggttggccaatatgattcttatcagggagctaattctaatgagctataccaaaatca
aaaccagttgtccatgatgtatcgaagtcaaagtgaattgcaaagtgatgattcagaggatgccccacccaaatcatggcatagtcgattaagcatt
gacctttctgataagactttcagcttcccaaaatttggatctacactgcagagggctaaatcagccttggaagtagtatggaacaaaagcacacaga
gtctgagtgggtatgaggacagtggctcttcattaatggggagatttcggacattatctcaatcaactgcaaatgagtcaagtaccacacttgactct
gatgtctacacggagccctattactataaagcagaggatgaggaagattatactgaaccagtggctgacaatgaaacagattatgttgaagtcatg
gaacaagtccttgctaaactagaaaacaggactagtattactgaaacagatgaacaaatgcaagcatatgatcacctttcatatgaaacaccttatga
aaccccacaagatgagggttatgatggtccagcagatgatatggttagtgaagaggggttagaacccttaaatgaaacatcagctgagatggaaa
taagagaagatgaaaaccaaaacattcctgaacagccagtggagatcacaaagccaaagagaattcgtccttctttcaaagaagcagctttaagg
gcctataaaaagcaaatggcagagttggaagagaagatcttggctggagatagcagttctgtggatgaaaaggctcgaatagtaagtggcaatg
atttggatgcttccaaattttctgcactccaggtgtgtggtggggctggaggtggactttatggtattgacagcatgccggatcttcgcagaaaaaaa
actttgcctattgtccgagatgtggccatgaccctggctgcccggaaatctggactctccctggctatggtgattaggacatccctaaataatgagg
aactgaaaatgcacgtcttcaagaagaccttgcaggcactgatctaccctatgtcttctaccatcccacacaattttgaggtctggacggctaccaca
cccacctactgttatgagtgtgaagggctcctgtggggcattgcaaggcaaggcatgaagtgtctggagtgtggagtgaaatgccacgaaaagt
gtcaggacctgctaaacgctgactgcttgcagagagcagcagaaaagagttctaaacatggtgccgaagacaagactcagaccattattacagc
aatgaaagaaagaatgaagatcagggagaaaaaccggccagaagtatttgaagtaatccaggaaatgtttcagatttctaaagaagattttgtgca
gtttacaaaggcggccaaacagagtgtactggatgggacatctaagtggtctgcaaaaataaccatcacagtggtttctgcacaagg SEQ ID NO:7)

TABLE 37
Predicted Amino Acid Seciuence of PN7098

MVANFFKSLILPYIHKLCKGMFTKKLGNTNKNKEYRQQKKDQDFPTAGQTKSPKFSYTFKS
TVKKIAKCSSTHNLSTEEDEASKEFSLSPTFSYRVAIANGLQKNAKVTTSDNEDLLQELSSIE
SSYSESLNELRSSTENQAQSTHTMPVRRNRKSSSSLAPSEGSSDGERTLHGLKLGALRKLRK
WKKSQECVSSDSELSTMKKSWGIRSKSLDRTVRNPKTNALEPGFSSSGCISQTHDVMEMIF
KELQGISQIETELSELRGHVNALKHSIDEISSSVEVVQSEIEQLRTGFVQSRRETRDIHDYIKH
LGHMGSKASLRFLNVTEERFEYVESVVYQILIDKMGFSDAPNAIKIEFAQRIGHQRDCPNAK
PRPILVYFETPQQRDSVLKKSYKLKGTGIGISTDILTHDIRERKEKGIPSSQTYESMAIKLSTPE
PKIKKNNWQSPDDSDEDLESDLNRNSYAVLSKSELLTKGSTSKPSSKSHSARSKNKTANSSR
ISNKSDYDKISSQLPESDILEKQTTTHYADATPLWHSQSDFFTAKLSRSESDFSKCQSYSED
FSENQFFTRTNGSSLLSSSDRELWQRKQEGTATLYDSPKDQHLNGSVQGIQGQTETENTETV
DSGMSNGMVCASGDRSHYSDSQLSLHEDLSPWKEWNQGADLGLDSSTQEGFDYETNSLFD
QQLDVYNKDLEYLGKCHSDLQDDSESYDLTQDDNSSPCPGLDNEPQGQWVGQYDSYQGA
NSNELYQNQNQLSMMYRSQSELQSDDSEDAPPKSWHSRLSIDLSDKTFSFPKFGSTLQRAK
SALEVVWNKSTQSLSGYEDSGSSLMGRFRTLSQSTANESSTTLDSDVYTEPYYYKAEDEED
YTEPVADNETDYVEVMEQVLAKLENRTSITETDEQMQAYDHLSYETPYETPQDEGYDGPA
DDMVSEEGLEPLNETSAEMEIREDENQNIPEQPVEITKPKRIRPSFKEAALRAYKKQMAELE
EKILAGDSSSVDEKARIVSGNDLDASKFSALQVCGGAGGGLYGIDSMPDLRRKKTLPIVRD
VAMTLAARKSGLSLAMVIRTSLNNEELKMHVFKKTLQALIYPMSSTIPHNFEVWTATTPTY
CYECEGLLWGIARQGMKCLECGVKCHEKCQDLLNADCLQRAAEKSSKHGAEDKTQTIITA
MKERMKIREKNRPEVFEVIQEMFQISKEDFVQFTKAAKQSVLDGTSKWSAKITITVVSAQX
(SEQ ID NO:8)

### EXAMPLES 6-32

### Identification of Protein-Protein Interactions

A yeast two-hybrid system as described in Example 1 using amino acids of the bait as set forth in Table 38 was performed. The clone that was identified by this procedure for each bait is set forth in Table 38 as the prey. The “AA” refers to the amino acids of the bait or prey. The “NUC” refers to the nucleotides of the bait or prey. The Accession numbers refer to GB: GenBank and SP: Swiss Protein accession numbers.

TABLE 38

| Ex. | BAIT | ACCESSION | COORDINATES | PREY | ACCESSION | COORDINATES |
|---|---|---|---|---|---|---|
| 6 | p38 ALPHA | SP: Q13083 | AA 1-130 | JNK3 ALPHA2 | SP: P53779 | AA 371-464 |
| 7 | p38 ALPHA | SP: Q13083 | AA 1-130 | C-NAP1 | GB: AF049105 | AA 1362-1579 |
| 8 | p38 ALPHA | SP: Q13083 | AA 194-319 | VINCULIN | SP: P18206 | AA 933-1067 |
| 9 | p38 ALPHA (K53M MUTANT) | SP: Q13083 | AA 1-361 | SPLICING FACTOR PSF | SP: P23246 | AA 282-577 |
| 10 | MAPKAP-K2 | SP: P49137 | AA 238-325 | LEUCINE-RICH PROTEIN L130 | SP: P42704 | AA 31-263 |
| 11 | MAPKAP-K2 | SP: P49137 | AA 134-325 | cAMP-DEP PROTEIN KINASE | SP: P10644 | AA 20-382 |
| 12 | MAPKAP-K2 | SP: P49137 | AA 134-325 | SET | SP: Q01105 | AA 106-239 |
| 13 | MAPKAP-K2 | SP: P49137 | AA 1-338 | TL21 | GB: X75692 | NUC 6-276 |
| 14 | MAPKAP-K2 (K93M, | SP: P49137 | AA 1-338 | ERK3 | SP: Q16659 | AA 19-509 |
| | T222D,T334D) | | | | | |
| 15 | MAPKAP-K3 | GB: U09578 | AA 1-304 | THROMBOSPONDIN 3 | SP: P49746 | AA 215-366 |
| 16 | MAPKAP-K3 | GB: U09578 | AA 114-304 | MALATE DEHYDROGENASE | SP: P40925 | AA 1-326 |
| 17 | MAPKAP-K3 | GB: U09578 | AA 114-304 | GA17 | GB: AF064603 | AA 1-363 |
| 18 | MAPKAP-K3 | GB: U09578 | AA 114-304 | CALPAIN 4 SMALL SUBUNIT | SP: P04632 | AA 102-269 |
| 19 | MAPKAP-K3 | GB: U09578 | AA 217-304 | BAT3 | SP: P46379 | AA 190-473 |
| 20 | MSK-1 | GB: AF074393 | AA 426-686 | ABLIM | GB: AF005654 | AA 197-340 |
| 21 | MSK-1 | GB: AF074393 | AA 426-686 | KIAA0144 | SP: Q14157 | AA 690-857 |
| 22 | PRAK | GB: AF032437 | AA 3-304 | KENDRIN | GB: U52962 | AA 191-574 |
| 23 | PRAK | GB: AF032437 | AA 3-304 | HOMEOTIC PROTEIN PROX1 | SP: Q92786 | AA 203-464 |
| 24 | PRAK | GB: AF032437 | AA 3-304 | HOOK1 | GB: | AA 1-413 |
| | | | | | AF0414923 | |
| 25 | PRAK | GB: AF032437 | AA 3-304 | IG HEAVY CHAIN CONSTANT | SP: P01857 | AA 105-192 |
| | | | | REGION | | |
| 26 | PRAK | GB: AF032437 | AA 3-304 | GOLGIN-95 | SP: Q08379 | AA 22-482 |
| 27 | PRAK | GB: AF032437 | AA 3-304 | KIAA0555 | GB: AB011127 | AA 461-583 |
| 28 | PRAK | GB: AF032437 | AA 198-304 | LEUCINE-RICH PROTEIN L130 | SP: P42704 | AA 31-263 |
| 29 | PRAK | GB: AF032437 | AA 304-471 | ERK3 | SP: Q16659 | AA 36-502 |
| 30 | PRAK | GB: AF032437 | AA 304-471 | cAMP-DEP PROTEIN KINASE | SP: P10644 | AA 19-141 |
| 31 | PRAK | GB: AF032437 | AA 3-304 | AL117538 | GB: AL117538 | AA 1-43 |
| 32 | PRAK | GB: AF032437 | AA 3-304 | AL117237 | GB: AL117237 | AA 401-488 |

### Example 33

### Generation of Polyclonal Antibody Against Protein Complexes

As shown above, p38 alpha interacts with CYT4 to form a complex. A complex of the two proteins is prepared, e.g., by mixing purified preparations of each of the two proteins. If desired, the protein complex can be stabilized by cross-linking the proteins in the complex, by methods known to those of skill in the art. The protein complex is used to immunize rabbits and mice using a procedure similar to that described by Harlow et al. (1988). This procedure has been shown to generate Abs against various other proteins (for example, see Kraemer et al., 1993).

Briefly, purified protein complex is used as immunogen in rabbits. Rabbits are immunized with 100 μg of the protein in complete Freund's adjuvant and boosted twice in three-week intervals, first with 100 μg of immunogen in incomplete Freund's adjuvant, and followed by 100 μg of immunogen in PBS. Antibody-containing serum is collected two weeks thereafter. The antisera is preadsorbed with P38 alpha and CYT4, such that the remaining antisera comprises antibodies which bind conformational epitopes, i.e., complex-specific epitopes, present on the P38 alpha-CYT4 complex but not on the monomers.

Polyclonal antibodies against each of the complexes set forth in Tables 1-31 are prepared in a similar manner by mixing the specified proteins together, immunizing an animal and isolating antibodies specific for the protein complex, but not for the individual proteins.

Polyclonal antibodies against each of the proteins set forth in Tables 33, 35 and 37 are prepared in a similar manner by immunizing an animal with the protein and isolating antibodies specific for the protein.

### Example 34

### Generation of Monoclonal Antibodies Specific for Protein Complexes

Monoclonal antibodies are generated according to the following protocol. Mice are immunized with immunogen comprising P38 alpha/CYT4 complexes conjugated to keyhole limpet hemocyanin using glutaraldehyde or EDC as is well known in the art. The complexes can be prepared as described in Example 33, and may also be stabilized by cross-linking. The immunogen is mixed with an adjuvant. Each mouse receives four injections of 10 to 100 μg of immunogen, and after the fourth injection blood samples are taken from the mice to determine if the serum contains antibody to the immunogen. Serum titer is determined by ELISA or RIA. Mice with sera indicating the presence of antibody to the immunogen are selected for hybridoma production.

Spleens are removed from immune mice and a single-cell suspension is prepared (Harlow et al., 1988). Cell fusions are performed essentially as described by Kohler et al. (1975). Briefly, P3.65.3 myeloma cells (American Type Culture Collection, Rockville, Md.) or NS-1 myeloma cells are fused with immune spleen cells using polyethylene glycol as described by Harlow et al. (1988). Cells are plated at a density of 2×10⁵ cells/well in 96-well tissue culture plates. Individual wells are examined for growth, and the supernatants of wells with growth are tested for the presence of P38 alpha/CYT4 complex-specific antibodies by ELISA or RIA using P38 alpha/CYT4 complex as target protein. Cells in positive wells are expanded and subcloned to establish and confirm monoclonality.

Clones with the desired specificities are expanded and grown as ascites in mice or in a hollow fiber system to produce sufficient quantities of antibodies for characterization and assay development. Antibodies are tested for binding to P38 alpha alone or to CYT4 alone, to determine which are specific for the P38 alpha/CYT4 complex as opposed to those that bind to the individual proteins.

Monoclonal antibodies against each of the complexes set forth in Tables 1-31 are prepared in a similar manner by mixing the specified proteins together, immunizing an animal, fusing spleen cells with myeloma cells and isolating clones which produce antibodies specific for the protein complex, but not for the individual proteins.

Monoclonal antibodies against each of the proteins set forth in Tables 33, 35 and 37 are prepared in a similar manner by immunizing an animal with the protein, fusing spleen cells with myeloma cells and isolating clones which produce antibodies specific for the protein.

### Example 35

### In vitro Identification of Modulators for Protein-Protein Interactions

The present invention is useful in screening for agents that modulate the interaction of P38 alpha and CYT4. The knowledge that P38 alpha and CYT4 form a complex is useful in designing such assays. Candidate agents are screened by mixing P38 alpha and CYT4 (a) in the presence of a candidate agent, and (b) in the absence of the candidate agent. The amount of complex formed is measured for each sample. An agent modulates the interaction of P38 alpha and CYT4 if the amount of complex formed in the presence of the agent is greater than (promoting the interaction), or less than (inhibiting the interaction) the amount of complex formed in the absence of the agent. The amount of complex is measured by a binding assay, which shows the formation of the complex, or by using antibodies immunoreactive to the complex.

Briefly, a binding assay is performed in which immobilized P38 alpha is used to bind labeled CYT4. The labeled CYT4 is contacted with the immobilized P38 alpha under aqueous conditions that permit specific binding of the two proteins to form a P38 alpha/CYT4 complex in the absence of an added test agent. Particular aqueous conditions may be selected according to conventional methods. Any reaction condition can be used as long as specific binding of P38 alpha/CYT4 occurs in the control reaction. A parallel binding assay is performed in which the test agent is added to the reaction mixture. The amount of labeled CYT4 bound to the immobilized P38 alpha is determined for the reactions in the absence or presence of the test agent. If the amount of bound, labeled CYT4 in the presence of the test agent is different than the amount of bound labeled CYT4 in the absence of the test agent, the test agent is a modulator of the interaction of P38 alpha and CYT4.

Candidate agents for modulating the interaction of each of the protein complexes set forth in Tables 1-31 are screened in vitro in a similar manner.

### Example 36

### In vivo Identification of Modulators for Protein-Protein Interactions

In addition to the in vitro method described in Example 35, an in vivo assay can also be used to screen for agents which modulate the interaction of P38 alpha and CYT4. Briefly, a yeast two-hybrid system is used in which the yeast cells express (1) a first fusion protein comprising P38 alpha or a fragment thereof and a first transcriptional regulatory protein sequence, e.g., GAL4 activation domain, (2) a second fusion protein comprising CYT4 or a fragment thereof and a second transcriptional regulatory protein sequence, e.g., GAL4 DNA-binding domain, and (3) a reporter gene, e.g., β-galactosidase, which is transcribed when an intermolecular complex comprising the first fusion protein and the second fusion protein is formed. Parallel reactions are performed in the absence of a test agent as the control and in the presence of the test agent. A functional P38 alpha/CYT4 complex is detected by detecting the amount of reporter gene expressed. If the amount of reporter gene expression in the presence of the test agent is different than the amount of reporter gene expression in the absence of the test agent, the test agent is a modulator of the interaction of P38 alpha and CYT4.

Candidate agents for modulating the interaction of each of the protein complexes set forth in Tables 1-31 are screened in vivo in a similar manner.

While the invention has been disclosed in this patent application by reference to the details of preferred embodiments of the invention, it is to be understood that the disclosure is intended in an illustrative rather than in a limiting sense, as it is contemplated that modifications will readily occur to those skilled in the art, within the spirit of the invention and the scope of the appended claims.

### LIST OF REFERENCES

Aronheim et al., 1997. Isolation of an AP-1 repressor by a novel method for detecting protein-protein interactions. Mol. Cell. Biol. 17:3094-3102.

Augustin, I. et al. (1999). Differential expression of two novel Munc13 proteins in rat brain. Biochem J. 337 (Pt 3):363-71.

Altschul, S. F. et al. (1990). Basic local alignment search tool. J. Mol. Biol. 215:403-410.

Altschul, S. F. et al. (1997). Gapped BLAST and PSI-BLAST: a new generation of protein database search programs. Nucl. Acids Res. 25:3389-3402.

Banerji, J. et al. (1990). A gene pair from the human major histocompatibility complex encodes large proline-rich proteins with multiple repeated motifs and a single ubiquitin-like domain. Proc Natl Acad Sci USA 87:2374-8.

Barr, F. A. (1999). A novel Rab6-interacting domain defines a family of Golgi-targeted coiled-coil proteins. Curr Biol. 9:381-4.

Bartel, P. L. et al. (1993). “Using the 2-hybrid system to detect protein-protein interactions.” In: Cellular Interactions in Development: A Practical Approach, Oxford University Press, pp. 153-179.

Bartel, P. L. et al. (1996). A protein linkage map of Escherichia coli bacteriophage T7. Nat Genet 12:72-77.

Bartel, P. L. and Fields, S. (1997). The Yeast Two-Hybrid System. New York: Oxford University Press.

Blok, L. J. et al. (1995). Isolation of cDNAs that are differentially expressed between androgen-dependent and androgen-independent prostate carcinoma cells using differential display PCR. Prostate 26:213-24.

Bouckson-Castaing, V. et al. (1996). Molecular characterisation of ninein, a new coiled-coil protein of the centrosome. J Cell Sci. 109 (Pt 1): 179-90.

Carlson, S. et al. (1998). Expression of SET, an inhibitor of protein phosphatase 2A, in renal development and Wilms' tumor. J. Am. Soc. Nephrol. 9:1873-1880.

Chardin, P. et al. (1996). A human exchange factor for ARF contains Sec7- and pleckstrin-homology domains. Nature 384:481-4.

Cheng, M. et al. (1996). ERK3 is a constitutively nuclear protein kinase. J Biol Chem. 271:8951-8.

Chevray, P. M. and Nathans, D. N. (1992). Protein interaction cloning in yeast: identification of mammalian proteins that interact with the leucine zipper of Jun. Proc. Natl. Acad. Sci. USA 89:5789-5793.

Daniel, J. M. et al. (1999). The catenin p120(ctn) interacts with Kaiso, a novel BTB/POZ domain zinc finger transcription factor. Mol Cell Biol. 19:3614-23.

Fields S and Song O -K (1989). A novel genetic system to detect protein-protein interactions. Nature 340:245-246.

Francis, S. H. et al. (1999). Cyclic nucleotide-dependent protein kinases: intracellular receptors for cAMP and cGMP action. Crit Rev Clin Lab Sci. 36:275-328.

Fritzler, M. J. et al. (1993). Molecular characterization of two human autoantigens: unique cDNAs encoding 95- and 160-kD proteins of a putative family in the Golgi complex. J Exp Med. 178:49-62.

Fry, A. M. et al. (1998). C-Napl, a novel centrosomal coiled-coil protein and candidate substrate of the cell cycle-regulated protein kinase Nek2. J Cell Biol. 141:1563-74.

Grammer, A. C. et al. (1998). TNF receptor-associated factor-3 signaling mediates activation of p38 and Jun N-terminal kinase, cytokine secretion, and Ig production following ligation of CD40 on human B cells. J Immunol. 161:1183-93.

Gupta, S. et al. (1996). Selective interaction of JNK protein kinase isoforms with transcription factors. EMBO J 15:2760-70.

Harlow et al. (1988). Antibodies: A Laboratory Manual (Cold Spring Harbor Laboratory, Cold Spring Harbor, N.Y.).

Hirano, K. et al. (1996). Interaction of protein phosphatase type 1 with a splicing factor. FEBS Lett. 389:191-4.

Hong, Y. R. et al. (2000). Cloning and characterization of a novel human ninein protein that interacts with the glycogen synthase kinase 3beta. Biochim Biophys Acta 1492:513-6.

Hou, J. et al. (1994). Molecular cloning and expression of the gene for a major leucine-rich protein from human hepatoblastoma cells (HepG2). In Vitro Cell Dev Biol Anim. 30A:111-4.

Kawasaki, H. et al. (1997). Activation and involvement of p38 mitogen-activated protein kinase in glutamate-induced apoptosis in rat cerebellar granule cells. J Biol Chem. 272:18518-21.

Kirchner, J. et al. (1999). Universal and unique features of kinesin motors: insights from a comparison of fungal and animal conventional kinesins. J. Biol Chem. 380:915-21.

Kohler, G. and Milstein, C. (1975). Continuous cultures of fused cells secreting antibody of predefined specificity. Nature 256:495-497.

Kouba, D. J. et al. (2000). Tumor necrosis factor-a induces distinctive NF-kappaB signaling within human dermal fibroblasts. J Biol Chem. (published electronically on Nov. 21, 2000 ahead of print).

Kraemer, F. B. et al. (1993). Detection of hormone-sensitive lipase in various tissues. I. Expression of an HSL/bacterial fusion protein and generation of anti-HSL antibodies. J. Lipid Res. 34:663-672.

Kramer, H. et al. (1996). Mutations in the Drosophila hook gene inhibit endocytosis of the boss transmembrane ligand into multivesicular bodies. J Cell Biol. 133:1205-15.

Leanna, C. A. and Hannink, M. (1996). The reverse two-hybrid system: a genetic scheme for selection against specific protein/protein interactions. Nucl. Acids Res. 24:3341-3347.

Lee, S. C. et al. (1994). A protein kinase involved in the regulation of inflammatory cytokine biosynthesis. Nature 372:739-46.

Lee, S. C. et al. (1996). Role of CSB/p38/RK stress response kinase in LPS and cytokine signaling mechanisms. J Leukoc Biol. 59:152-7.

Li, M. et al. (1996). The myeloid leukemia-associated protein SET is a potent inhibitor of protein phosphatase 2A.: J. Biol. Chem. 271:11059-11062.

Mach, F. et al. (1998). Reduction of atherosclerosis in mice by inhibition of CD40 signaling. Nature 394:200-3.

Mack, G. J. et al. (1998). Autoantibodies to a group of centrosomal proteins in human autoimmune sera reactive with the centrosome. Arthritis Rheum. 41:551-8.

Miyazawa, K. et al. (1998). Regulation of interleukin-1beta-induced interleukin-6 gene expression in human fibroblast-like synoviocytes by p38 mitogen-activated protein kinase. J Biol Chem. 273:24832-8.

Musrati, R. A. et al. (1998). Malate dehydrogenase: distribution, function and properties. Gen Physiol Biophys. 17:193-210.

Nemoto, S. et al. (1998). Opposing effects of Jun kinase and p38 mitogen-activated protein kinases on cardiomyocyte hypertrophy. Mol Cell Biol. 18:3518-26.

Noguchi, M. et al. (1997). Functional cleavage of the common cytokine receptor gamma chain (gammac) by calpain. Proc Natl Acad Sci USA 94:11534-9.

Ogasawara, M. et al. (2000). Similarities in function and gene structure of cytohesin-4 and cytohesin-1, guanine nucleotide-exchange proteins for ADP-ribosylation factors. J Biol Chem 275:3221-30.

Ozaki, T. et al. (1999). Cloning and characterization of rat BAT3 cDNA. DNA Cell Biol. 18:503-12.

Patton, J. G. et al. (1993). Cloning and characterization of PSF, a novel pre-mRNA splicing factor. Genes Dev. 7:393-406.

Purohit, A. et al. (1999). Direct interaction of pericentrin with cytoplasmic dynein light intermediate chain contributes to mitotic spindle organization. J Cell Biol. 147:481-92.

Qabar, A. N. et al. (1994). Thrombospondin 3 is a developmentally regulated heparin binding protein. J Biol Chem. 269:1262-9.

Raingeaud, J. et al. (1995). Pro-inflammatory cytokines and environmental stress cause p38 mitogen-activated protein kinase activation by dual phosphorylation on tyrosine and threonine. J Biol Chem. 270:7420-6.

Roof, D. J. et al. (1997). Molecular characterization of abLIM, a novel actin-binding and double zinc finger protein. J Cell Biol. 138:575-88.

Rudiger, M. (1998). Vinculin and alpha-catenin: shared and unique functions in adherens junctions. Bioessays 20:733-40.

Saido, T. C. et al. (1994). Calpain: new perspectives in molecular diversity and physiological-pathological involvement. FASEB J 8:814-22.

Vaesen, M. et al. (1994). Purification and characterization of two putative HLA class II associated proteins: PHAPI and PHAPII. Biol. Chem. Hoppe-Seyler 375:113-126.

von Lindem, M. et al. (1992). Can, a putative oncogene associated with myeloid leukemogenesis, may be activated by fusion of its 3′ half to different genes: characterization of the set gene. Mol Cell Biol. 12:3346-55.

Wang, Y. et al. (1998). Cardiac muscle cell hypertrophy and apoptosis induced by distinct members of the p38 mitogen-activated protein kinase family. J Biol Chem. 273:2161-8.

Wetmur, J. G. and Davidson, N. (1968). “Kinetics of renaturation of DNA.” J. Mol. Biol. 31:349-370.

Wigle, J. T. et al. (1999). Proxi function is required for the development of the murine lymphatic system. Cell 98:769-78.

PCT Published Application No. WO 97/27296

PCT Published Application No. WO 99/65939

U.S. Pat. No. 5,622,852

U.S. Pat. No. 5,773,218

What is claimed is:

1. An isolated protein complex comprising two proteins, the protein complex selected from the group consisting of
(a) a complex set forth in Table 1;
(b) a complex set forth in Table 2;
(c) a complex set forth in Table 3;
(d) a complex set forth in Table 4;
(d) a complex set forth in Table 5;
(d) a complex set forth in Table 6;
(d) a complex set forth in Table 7;
(d) a complex set forth in Table 8;
(d) a complex set forth in Table 9;
(d) a complex set forth in Table 10;
(d) a complex set forth in Table 11;
(d) a complex set forth in Table 12;
(d) a complex set forth in Table 13;
(d) a complex set forth in Table 14;
(d) a complex set forth in Table 15;
(d) a complex set forth in Table 16;
(d) a complex set forth in Table 17;
(d) a complex set forth in Table 18;
(d) a complex set forth in Table 19;
(d) a complex set forth in Table 20;
(d) a complex set forth in Table 21;
(d) a complex set forth in Table 22;
(d) a complex set forth in Table 23;
(d) a complex set forth in Table 24;
(d) a complex set forth in Table 25;
(d) a complex set forth in Table 26;
(d) a complex set forth in Table 27;
(d) a complex set forth in Table 28;
(d) a complex set forth in Table 29;
(d) a complex set forth in Table 30;
(d) a complex set forth in Table 31; and
(d) a complex set forth in Table 32.

2. The protein complex of claim 1, wherein said protein complex comprises complete proteins.

3. The protein complex of claim 1, wherein said protein complex comprises a fragment of one protein and a complete protein of anther protein.

4. The protein complex of claim 1, wherein said protein complex comprises fragments of proteins.

5. An isolated antibody selectively immunoreactive with a protein complex of claim 1.

6. The antibody of claim 5, wherein said antibody is a monoclonal antibody.

7. A method for diagnosing a physiological disorder in an animal, which comprises assaying for:
(a) whether a protein complex set forth in any one of Tables 1-31 is present in a tissue extract;
(b) the ability of proteins to form a protein complex set forth in any one of Tables 1-31; and
(c) a mutation in a gene encoding a protein of a protein complex set forth in any one of Tables 1-31.

8. The method of claim 7, wherein said animal is a human.

9. The method of claim 7, wherein the diagnosis is for a predisposition to said physiological disorder.

10. The method of claim 7, wherein the diagnosis is for the existence of said physiological disorder.

11. The method of claim 7, wherein said assay comprises a yeast two-hybrid assay.

12. The method of claim 7, wherein said assay comprises measuring in vitro a complex formed by combining the proteins of the protein complex, said proteins isolated from said animal.

13. The method of claim 12, wherein said complex is measured by binding with an antibody specific for said complex.

14. The method of claim 7, wherein said assay comprises mixing an antibody specific for said protein complex with a tissue extract from said animal and measuring the binding of said antibody.

15. A method for determining whether a mutation in a gene encoding one of the proteins of a protein complex set forth in any one of Tables 1-31 is useful for diagnosing a physiological disorder, which comprises assaying for the ability of said protein with said mutation to form a complex with the other protein of said protein complex, wherein an inability to form said complex is indicative of said mutation being useful for diagnosing a physiological disorder.

16. The method of claim 15, wherein said gene is an animal gene.

17. The method of claim 16, wherein said animal is a human.

18. The method of claim 15, wherein the diagnosis is for a predisposition to a physiological disorder.

19. The method of claim 15, wherein the diagnosis is for the existence of a physiological disorder.

20. The method of claim 15, wherein said assay comprises a yeast two-hybrid assay.

21. The method of claim 15, wherein said assay comprises measuring in vitro a complex formed by combining the proteins of the protein complex, said proteins isolated from an animal.

22. The method of claim 21, wherein said animal is a human.

23. The method of claim 21, wherein said complex is measured by binding with an antibody specific for said complex.

24. A method for screening for drug candidates capable of modulating the interaction of the proteins of a protein complex set forth in any one of Tables 1-31, which comprises:
(a) combining the proteins of said protein complex in the presence of a drug to form a first complex;
(b) combining the proteins in the absence of said drug to form a second complex;
(c) measuring the amount of said first complex and said second complex; and
(d) comparing the amount of said first complex with the amount of said second complex,
wherein if the amount of said first complex is greater than, or less than the amount of said second complex, then the drug is a drug candidate for modulating the interaction of the proteins of said protein complex.

25. The method of claim 24, wherein said screening is an in vitro screening.

26. The method of claim 24, wherein said complex is measured by binding with an antibody specific for said protein complexes.

27. The method of claim 24, wherein if the amount of said first complex is greater than the amount of said second complex, then said drug is a drug candidate for promoting the interaction of said proteins.

28. The method of claim 24, wherein if the amount of said first complex is less than the amount of said second complex, then said drug is a drug candidate for inhibiting the interaction of said proteins.

29. A non-human animal model for a physiological disorder wherein the genome of said animal or an ancestor thereof has been modified such that the formation of a protein complex set forth in any one of Tables 1-31 has been altered.

30. The non-human animal model of claim 29, wherein the formation of said protein complex has been altered as a result of:
(a) over-expression of at least one of the proteins of said protein complex;
(b) replacement of a gene for at least one of the proteins of said protein complex with a gene from a second animal and expression of said protein;
(c) expression of a mutant form of at least one of the proteins of said protein complex;
(d) a lack of expression of at least one of the proteins of said protein complex; or
(e) reduced expression of at least one of the proteins of said protein complex.

31. A cell line obtained from the animal model of claim 29.

32. A non-human animal model for a physiological disorder, wherein the biological activity of a protein complex set forth in any one of Tables 1-31 has been altered.

33. The non-human animal model of claim 32, wherein said biological activity has been altered as a result of:
(a) disrupting the formation of said complex; or
(b) disrupting the action of said complex.

34. The non-human animal model of claim 32, wherein the formation of said complex is disrupted by binding an antibody to at least one of the proteins which form said protein complex.

35. The non-human animal model of claim 32, wherein the action of said complex is disrupted by binding an antibody to said complex.

36. The non-human animal model of claim 32, wherein the formation of said complex is disrupted by binding a small molecule to at least one of the proteins which form said protein complex.

37. The non-human animal model of claim 32, wherein the action of said complex is disrupted by binding a small molecule to said complex.

38. A cell in which the genome of cells of said cell line has been modified to produce at least one protein complex set forth in any one of Tables 1-31.

39. A cell line in which the genome of the cells of said cell line has been modified to eliminate at least one protein of a protein complex set forth in any one of Tables 1-31.

40. A method of screening for drug candidates useful in treating a physiological disorder which comprises the steps of:
(a) measuring the activity of a protein selected from the proteins set forth in Tables 1-31 in the presence of a drug,
(b) measuring the activity of said protein in the absence of said drug, and
(c) comparing the activity measured in steps (1) and (2),
wherein if there is a difference in activity, then said drug is a drug candidate for treating said physiological disorder.

41. An isolated nucleic acid comprising a nucleic acid coding for a protein comprising an amino acid sequence selected from the group of amino acid sequences set forth in SEQ ID NOs:4, 6 and 8 and amino acid sequences having at least 95% identity to the amino acid sequences set forth in SEQ ID NOs:4, 6 and 8.

42. The nucleic acid of claim 41 wherein the nucleic acid comprises a nucleotide sequence selected from the group of nucleotide sequences set forth in SEQ ID NOs:3, 5 and 7, nucleotide sequences having at least 95% identity to the nucleotide sequences set forth in SEQ ID NOs:3, 5 and 7 and their complements.

43. A substantially pure protein comprising an amino acid sequence selected from the group of amino acid sequences set forth in SEQ ID NOs:4, 6 and 8 and amino acid sequences having al least 95% identity to the amino acid sequences set forth in SEQ ID NOs:4, 6 and 8.

44. An antibody specific for the protein of claim 43.

45. The antibody of claim 44 which is a monoclonal antibody.


SEQUENCE LISTING (8 sequences)

SEQ ID NO: 1 - DNA, 40 bp, Primer for yeast two-hybrid assays
gcaggaaacagctatgaccatacagtcagcggccgccacc

SEQ ID NO: 2 - DNA, 39 bp, Primer for yeast two-hybrid assays
acggccagtcgcgtggagtgttatgtcatgcggccgcta

SEQ ID NO: 3 - DNA, 5153 bp, Homo sapiens
  CDS (141)..(2159)
gccgcgtcgacgtcgacccagactggagcgacgtttaaagaaggggcagaatcgctggggagtgcggcttcttcttgttgggggactcccagccttccgcgcgtccggaggaggagaagcggcggcgccgggaagcaggcatggagagtagaaaactgatttctgctacagacattcagtactctggcagtctgctgaactccttgaatgagcaacgtggccatggactcttctgtgatgttaccgttattgtggaagaccgaaaattccgggctcacaagaatattctttcagcttctagtacctacttccatcagctcttctctgttgctgggcaagttgttgaactgagctttataagagcagagatctttgcagaaattctcaattatatctatagttctaaaattgttcgtgttagatcagatttgcttgatgagttaattaaatcagggcagttattaggagtgaaatttatagcagagcttggtgtcccattgtcacaggttaaaagcatctcaggtacagcgcaggatggtaatactgagcctttacctcctgattctggtgacaagaaccttgtaatacagaaatcaaaagatgaagcccaagataatggggctactataatgcctattataacagagtctttttcattatctgccgaagattatgaaatgaaaaagatcattgttaccgattctgatgatgatgatgatgatgatgtcattttttgctccgagattctgcccacaaaggagactttgccgagtaataacacagtggcacaggtccaatctaacccaggccctgttgctatttcagatgttgcacctagtgctagcaataactcgccccctttaacaaatatcacacctactcagaaacttcctactcctgtgaatcaggcaactttgagccaaacacaaggaagtgaaaaattgttggtatcttcagctccaacacatctgactcccaatattattttgttaaatcagacaccactttctacaccaccaaatgtcagttcttcacttccaaatcatatgccctcttcaatcaatttacttgtgcagaatcagcagacaccaaacagtgctattttaacaggaaacaaggccaatgaagaggaggaggaggaaataatagatgatgatgatgacactattagctccagtcctgactcggccgtcagtaatacatctttggtcccacaggctgatacctcccaaaataccagttttgatggatcattaatacagaagatgcagattcctacacttcttcaagaaccactttccaattccttaaaaatttcagatataattactagaaatactaatgatccaggcgtaggatcaaaacatctaatggagggtcagaagatcattactttagatacagctactgaaattgaaggcttatcgactggttgcaaggtttatgcaaatatcggtgaagatacttatgatatagtgatccctgtcaaagatgaccctgatgaaggggaggccagacttgagaatgaaataccaaaaacgtctggcagcgagatggcaaacaaacgtatgaaagtaaaacatgatgatcactatgagttaatagtagatggaagggtctattatatctgtattgtatgcaaaaggtcatatgtctgtctgacaagcttgcggagacattttaacattcattcttgggagaagaagtatccgtgccgttactgtgagaaggtatttcctcttgcagaatatcgcacaaagcatgaaattcatcacacaggggagcgaaggtatcagtgtttggcctgtggcaaatctttcatcaactatcagtttatgtcttcacatataaagtcagttcatagtcaagatccttctggggactcaaagctttatcgtttacatccatgcaggtctttacaaatcagacaatatgcatatctttccgatagatcaagcactattcctgcaatgaaggatgatggtattgggtataaggttgacactggaaaagaacctccagtagggaccactacatctactcagaacaagccaatgacctgggaagatatttttattcagcaggaaaatgattcaatttttaaacaaaatgtaacagatggcagtactgagtttgaatttataataccagagtcttactaaactcctttgaaatactagaaagttttgttttggatgatggggcaggggtttcagaagatctgtaaaacaaattaaggtgcgaacaagttaatttgatctgccacattatctgaaggaagtgtagtgggatttttgttgataatttttagaagcaaattttcctgaaagttttgagtagaggtgagaccccctccccaagtatctgtttatatagttagttttcagctcatttaaaagaggcaaaaattaaaagcttggagagatagtttcctgaatagaatttgaagcagtctgaatgttctttgaaaataactggagttattagcataccctagtacatcttacagctttccccttccatgttagcactttactgctgaattctcaattttcttaacattgagacaataaatgtgtgttttgtcttgtatatggcataaagagtaaataagttttagagttgttctggaaaatgtcagaataagtcagtacttgggttgtgtaatctgctagtccaagcgaacagcaacctcctgctaccctccctctatgaaaatagccatgcagacaagtctctcatctgaagaacaaattagatttagctaattagaattaatcctggctttcattgccatagtctgtaaaagactttggtggctagaccactttatacctttgcagtgtggtctctgggggcaaaaaactaatgaaaacaatctctgtaatggcagataggaggagatgaaaagttctgttgcatggatttttaattctctggctaccacatagtagagaatggaatgaagatttccttttggcttcttaaggttaaaaatattcccatgaacatgaaaattttcaaattttgaatctgaaagccaccaaatgtatctttatgtataaatccttgtaaatgatagattccatgggtgagactttacatattttgggtgggaggctactggcatatatttttaaatgttcatattgcgtagaatctccactaggaagtctttatttgaaatagttgaatcagtgatctagtattttcctttcggcaagatttgttaggtttttaccccttctaaaataagttttattccatctgcaaattgctgcaatattatagtaatcagaaactacataaggaatgttatataggcttgtcagttcccgtttttcttgacaacaataaataccacttttaaaaatgacacatatttaaacacttagaaaataaagttaacacttactgaagtgctagtactaaactgtgctagtactaaaagaaaacaggttggaacatacatatagcctagcatttataacagaattgttgaacgtctgtaaatgattttttttttttttgcaaaggaaaaaattgatactggaaaagattgttgtgcatagttattagtcatttgtaaccttgcttaagtatttcttagtccaacatagatattttctttctcctgaccatgtattttaaaatatagtctatttcttgactttgaacttaaagctttaatcataatttctcatgtatacatcgttcttctgatggtaagctggatttgaaggtagtggtttcagtgtttcttaagttggtagctgagggtatcaggcatcagttcatgcaataatacaagaaaaaaaatcctttgcttgccaagaggtagagtgatgtgcatttatctgttttctgttctgtaagtctagaccttcaaaccatttgtaaactaacccctgggaaatttgaaattacctgataacttaagactctgtgatctctggaatcaccatatgtttcttttttgtgtagatattaataacattactctttgactatagtgtgcactctgaaatgtactcagtgaaaatttgttttgagtttcattaatgctatttcaccagttagacataattacttctaccgatgtgaatgatacggatgccggcagagcttccagatctttcagactcaactgctaggtcaattagtttgtcataataaaacttggcagattctacaagtctattatgacaaaccaggaactaattctataatggaaaactatccattctgaataataggtatgtaattatttgctgctgctgctgtgctctgtaaattctgaatatgacatttaaactctgtgcctactaaaggtatcttctggagtttttgggaggagagaaactggaaaattaaattgtatttttgccagaagactcttacttgcatgtgtctcagggtcttcagtttttctataagtttccatatccaaagttcagaattcatgtgaaatacttctttggggcaaaagtccttcattcctggtatttattggattggaaatctgtagcaagatgctgtttaaaattaccatattgtttttttatcttatacttagctctctggctattgaacttccttttcttgtttgaagttagcttcaaatttgctcctatgctaaattacctgtaaatattctggataggaactacttgaaatagtaatttgttaaaagatatgacaaaatgaaaatgcttaaactacagaaatttaaaaatgccataacaatcttgcaagactaactttaaaatatactttaaatgattattatgattttggtggtaacgatcccccacacacaaccactatgaagaaataatgccgcatttttcccccattgtaccaaaaagataaaaaaatggtaaacactgatcaaggtattttgtattgtcaaggcatgcatattctaaagaattaaatgctaacttaacagcactggctttctggctggtcaactatatgaaaccttgttcattcctccgagtactgtaatgttcacacttgtacaatcttccctgtcatgactttaagttctacttttcattaaccatggcctgatattagttcttagagcttcttgtggcaaaaataaaatgatttaattctgaaaaaaaaaaaaaaaaaaaaaaaaaaaa

SEQ ID NO: 4 - protein, 673 aa, Homo sapiens
MESRKLISATDIQYSGSLLNSLNEQRGHGLFCDVTVIVEDRKFRAHKNILSASSTYFHQLFSVAGQVVELSFIRAEIFAEILNYIYSSKIVRVRSDLLDELIKSGQLLGVKFIAELGVPLSQVKSISGTAQDGNTEPLPPDSGDKNLVIQKSKDEAQDNGATIMPIITESFSLSAEDYEMKKIIVTDSDDDDDDDVIFCSEILPTKETLPSNNTVAQVQSNPGPVAISDVAPSASNNSPPLTNITPTQKLPTPVNQATLSQTQGSEKLLVSSAPTHLTPNIILLNQTPLSTPPNVSSSLPNHMPSSINLLVQNQQTPNSAILTGNKANEEEEEEIIDDDDDTISSSPDSAVSNTSLVPQADTSQNTSFDGSLIQKMQIPTLLQEPLSNSLKISDIITRNTNDPGVGSKHLMEGQKIITLDTATEIEGLSTGCKVYANIGEDTYDIVIPVKDDPDEGEARLENEIPKTSGSEMANKRMKVKHDDHYELIVDGRVYYICIVCKRSYVCLTSLRRHFNIHSWEKKYPCRYCEKVFPLAEYRTKHEIHHTGERRYQCLACGKSFINYQFMSSHIKSVHSQDPSGDSKLYRLHPCRSLQIRQYAYLSDRSSTIPAMKDDGIGYKVDTGKEPPVGTTTSTQNKPMTWEDIFIQQENDSIFKQNVTDGSTEFEFIIPESY

SEQ ID NO: 5 - DNA, 10625 bp, Homo sapiens
  CDS (544)..(6960)
cttattttgaaaacatttacatagtgattagttaacccaacagaccaatcctgggaagacagccagagcctgcagcaccttagtaacagaaaaactgataattaggagaagagacctgtccaagaccaggaacctggaccaaaattgtgccatgttgctttactttaatgagtggccccagtaaaaactgagctgtatggcagagctgttcacatttatcttctgtgtccacccagttctgctgaaacccctggcaagatcgtggccctgttgtagcttgtcatgttttgaacagctgtctatggaaagaaagcaaacacaacctagagcaacattgatttgttttagaaagctcttttattttcagttctggctgtgttcaacatcttagcttacgtttttcatgttgtaatgatctgccgtatggacgatcacctctaagttagagagttctgtaatttggcttggattaaagatgcttggttagtgaaagctgctgctttttttatagtcaaaggactggttctgagagccttgttgcagatggctgaggtcaccgtcccaagggtgtatgtcgtgtttggcatccattgcatcatggcgaaggcatcttcagatgtgcaggtttcaggctttcatcggaaaatccagcacgttaaaaatgaactttgccacatgttgagcttggaggaggtggccccagtgctgcagcagacattacttcaggacaacctcttgggcagggtacattttgaccaatttaaagaagcattaatactcatcttgtccagaactctgtcaaatgaagaacactttcaagaaccagactgctcactagaagctcagcccaaatatgttagaggtgggaagcgttacggacgaaggtccttgcccgagttccaagagtccgtggaggagtttcctgaagtgacggtgattgagccactggatgaagaagcgcggccttcacacatcccagccggtgactgcagtgagcactggaagacgcaacgcagtgaggagtatgaagcggaaggccagttaaggttttggaacccagatgacttgaatgcttcacagagtggatcttcccctccccaagactggatagaagagaaactgcaagaagtttgtgaagatttggggatcacccgtgatggtcacctgaaccggaagaagctggtctccatctgtgagcagtatggtttacagaatgtggatggagagatgctcgaggaagtattccataatcttgatcctgacggtacaatgagtgtagaagattttttctatggtttgtttaaaaatggaaaatctcttacaccatcagcatctactccatatagacaactaaaaaggcacctttccatgcagtctttcgatgagagtggacgacgtaccacaacctcatcagcaatgacaagtaccattggctttcgggtcttctcctgcctggatgatgggatgggccatgcatctgtggagagaatactggacacctggcaggaagagggcattgagaacagccaggagatcctgaaggccttggatttcagcctcgatggaaacatcaatttgacagaattaacactggcccttgaaaatgaacttttggttaccaagaacagcattcaccaggcggctctggccagctttaaggctgaaatccggcatttgttggaacgagttgatcaggtggtcagagaaaaagagaagctacggtcagatctggacaaggccgagaagctcaagtctttaatggcctcggaggtggatgatcaccatgcggccatagagcggcggaatgagtacaacctcaggaaactggatggagagtacaaggagcgaatagcagccttaaaaaatgaactccgaaaagagagagagcagatcctgcagcaggcaggcaagcagcgtttagaacttgaacaggaaattgaaaaggcaaaaacagaagagaactatatccgggaccgccttgccctctctttaaaggaaaacagtcgtctggaaaatgagcttctagaaaatgcagagaagttggcagaatatgagaatctgacaaacaaacttcagagaaatttggaaaatgtgttagcagaaaagtttggtgacctcgatcctagcagtgctgagttcttcctgcaagaagagagactgacacagatgagaaatgaatatgagcggcagtgcagggtactacaagaccaagtagatgaactccagtctgagctggaagaatatcgtgcacaaggcagagtgctcaggcttccgttgaagaactcaccgtcagaagaagttgaggctaacagcggtggcattgagcccgaacacgggctcggttctgaagaatgcaatccattgaatatgagcattgaggcagagctggtcattgaacagatgaaagaacaacatcacagggacatatgttgcctcagactggagctcgaagataaagtgcgccattatgaaaagcagctggacgaaaccgtggtcagctgcaagaaggcacaggagaacatgaagcaaaggcatgagaacgaaacgcgcaccttagaaaaacaaataagtgaccttaaaaatgaaattgctgaacttcaggggcaagcagcagtgctcaaggaggcacatcatgaggccacttgcaggcatgaggaggagaaaaaacaactgcaagtgaagcttgaggaggaaaagactcacctgcaggagaagctgaggctgcaacatgagatggagctcaaggctagactgacacaggctcaagcaagctttgagcgggagagggaaggccttcagagtagcgcctggacagaagagaaggtgagaggcttgactcaggaactagagcagtttcaccaggagcagctgacaagcctggtggagaaacacactcttgagaaagaggagttaagaaaagagctcttggaaaagcaccaaagggagcttcaggagggaagggaaaaaatggaaacagagtgtaatagaagaacctctcaaatagaagcccagtttcagtctgattgtcagaaagtcactgagaggtgtgaaagcgctctgcaaagcctggaggggcgctaccgccaagagctgaaggacctccaggaacagcagcgtgaggagaaatcccagtgggaatttgagaaggacgagctcacccaggagtgtgcggaagcccaggagctgctgaaagagactcttaagagagagaaaacaacttctctggtcctgacccaggagagagagatgctggagaaaacatacaaagaacatttgaacagcatggtcgtcgagagacagcagctactccaagacctggaagacctaagaaatgtatctgaaacccagcaaagcctgctgtctgaccagatacttgagctgaagagcagtcacaaaagggaactgagggagcgtgaggaggtcctgtgccaggcaggggcttcggagcagctggccagccagcggctggaaagactagaaatggaacatgaccaggaaaggcaggaaatgatgtccaagcttctagccatggagaacattcacaaagcgacctgtgagacagcagatcgagaaagagccgagatgagcacagaaatctccagacttcagagtaaaataaaggaaatgcagcaggcaacatctcctctctcaatgcttcagagtggttgccaggtgataggagaggaggaggtggaaggagatggagccctgtccctgcttcagcaaggggagcagctgttggaagaaaatggggacgtcctcttaagcctgcagagagctcatgaacaggcagtgaaggaaaatgtgaaaatggctactgaaatttctagattgcaacagaggctacaaaagttagagccagggttagtaatgtcttcttgtttggatgagccagctactgagttttttggaaatactgcggaacaaacagagcagtttttacagcaaaaccgaacgaagcaagtagaaggtgtgaccaggcggcatgtcctaagtgacctggaagatgatgaggtccgggacctgggaagtacagggacgagctctgttcagagacaggaagtcaaaatagaggagtctgaagcttcagtagagggtttttctgagcttgaaaacagtgaagagaccaggactgaatcctgggagctgaagaatcagattagtcagcttcaggaacagctaatgatgttatgtgcggactgtgatcgagcttctgaaaagaaacaggacctactttttgatgtttctgtgctaaaaaagaaactgaagatgcttgagagaatccctgaggcttctcccaaatataagctgttgtatgaagatgtgagccgagaaaatgactgccttcaggaagagctgagaatgatggagacacgctacgatgaggcactagaaaataacaaagaactcactgcagaggttttcaggttgcaggatgagctgaagaaaatggaggaagtcactgaaacattcctcagcctggaaaagagttacgatgaggtcaaaatagaaaatgaggggctgaatgttctggttttgagacttcaaggcaagattgagaagcttcaggaaagcgtggtccagcggtgtgactgctgcttatgggaagccagtttagagaacctggaaatcgaacctgatggaaatatactccagctcaatcagacactggaagagtgtgtgcccagggttaggagtgtacatcatgtcatagaggaatgtaagcaagaaaaccagtaccttgaggggaacacacagctcttggaaaaagtaaaagcacatgaaattgcctggttacatggaacaattcagacacatcaagaaaggccaagagtacagaatcaagttatactggaggaaaacactactctcctaggctttcaagacaaacattttcagcatcaggccaccatagcagagttagaactggagaaaacaaagttacaggagctgactaggaagttgaaggagagagtcactattttagttaagcaaaaagatgtactttctcacggagaaaaggaggaagagctgaaggcaatgatgcatgacttgcagatcacgtgcagtgagatgcagcaaaaagttgaacttctgagatatgaatctgaaaagcttcaacaggaaaattctattttgagaaatgaaattactactttaaatgaagaagatagcatttctaacctgaaattagggacattaaatggatctcaggaagaaatgtggcaaaaaacggaaactgtaaaacaagaaaatgctgcagttcagaagatggttgaaaatttaaagaaacagatttcagaattaaaaatcaaaaaccaacaattggatttggaaaatacagaacttagccaaaagaactctcaaaaccaggaaaaactgcaagaacttaatcaacgtctaacagaaatgctatgccagaaggaaaaagagccaggaaacagtgcattggaggaacgggaacaagagaagtttaatctgaaagaagaactggaacgttgtaaagtgcagtcctccactttagtgtcttctctggaggcggagctctctgaagttaaaatacagacccatattgtgcaacaggaaaaccaccttctcaaagatgaactggagaaaatgaaacagctgcacagatgtcccgatctctctgacttccagcaaaaaatctctagtgttctaagctacaacgaaaaactgctgaaagaaaaggaagctctgagtgaggaattaaatagctgtgtcgataagttggcaaaatcaagtcttttagagcatagaattgcgacgatgaagcaggaacagaaatcctgggaacatcagagtgcgagcttaaagtcacagctggtggcttctcaggaaaaggttcagaatttagaagacaccgtgcagaatgtaaacctgcaaatgtcccggatgaaatctgacctacgagtgactcagcaggaaaaggaggctttaaaacaagaagtgatgtctttacataagcaacttcagaatgctggtggcaagagctgggccccagagatagctactcatccatcagggctccataaccagcagaaaaggctgtcctgggacaagttggatcatctgatgaatgaggaacagcagctgctttggcaagagaatgagaggctccagaccatggtacagaacaccaaagccgaactcacgcactcccgggagaaggtccgtcaattggaatccaatcttcttcccaagcaccaaaaacatctaaacccatcaggtaccatgaatcccacagagcaagaaaaattgagcttaaagagagagtgtgatcagtttcagaaagaacaatctcctgctaacaggaaggtcagtcagatgaattcccttgaacaagaattagaaacaattcatttggaaaatgaaggcctgaaaaagaaacaagtaaaactggatgagcagctcatggagatgcagcacctgaggtccactgcgacgcctagcccgtcccctcatgcttgggatttgcagctgctccagcagcaagcctgtccgatggtgcccagggagcagtttctgcagcttcaacgccagctgctgcaggcagaaaggataaaccagcacctgcaggaggaacttgaaaacaggacctccgaaaccaacacaccacagggaaaccaggaacaactggtaactgtcatggaggaacgaatgatagaagttgaacagaaactgaaactagtgaaaaggcttcttcaagagaaagtgaatcagctcaaagaacaactctgcaagaacactaaggcagacgcaatggtgaaggacttgtatgttgaaaatgcccagttgttgaaagctctggaagtgactgaacagcgacagaaaacagcagagaagaaaaattacctcctggaggagaagattgccagcctcagtaatatagttaggaatctgacaccagcgccattgacttctacacctcctttgaggtcatagccaaaccaaagggtacactcatatttgtgcactttactgaaatagatgaacatttcagtaggttctcaacttaaaattaagcctaacctaaaactgccagcaacacaactggagtttccatttatcataattagtttttctaaatagacccttatgggagtttgaaaataaatactcacatatttcactacttaaattattcccaagatttgaatttattttaaaattttaatagccaccaagaatgtggacatatgaaaattcaagaacctaaaaaataccagttttgaatgagtttttgtggttttggttttttaattattacaaatctatgtgtaaaatctagatatttgaagtttgagatctgatgagaatggttgttataaactttattttaaaaccaaatttaggtgttcttacatatttaaatactggaaagtcattataatagttttggttctttgaattggtagacaattagtagagtataattggttaggaggcagggcttattaagtggttattaaccgctgacatcagacaaacccaaatctgtagaattctaacctcctaacacctgtgacagtattaccactcttcttgtattatagatttagaactgatttactcaattgcactcttaactaatgttaaaagcttacttgctttaaacagccttttcttctttctcttaaaagtttcatttggggagctggtcttctaagaaacggataaagccacataattaaagcagttgaactagagggaaagcactgaacaaaccactttggagtaaatagctactcttagaaaagagggataagcagaccatgtaggttttctgtctctcaaatcttagagttcataaatttacttgaggttgcctcaagaactcagggaacaatactgtaaactgtcttcctgaactactgtagggcctctctaagaatttgaaatgtataaaccatgtgacctcatttatttgtcttatatatttacagccatactagaatttttatttctacgtttttagtaaatttaatattctgggggaaaaaaggccttgattttagggttaaaaacctgacttatagaagagtttatttaatataggtcaaaattttctgtgtttcttattccttctatacctcaaatctgattctaagaatttcttactgtgataatcattggcatgccacctgaggtcaaggagtgccaaataggactttccactcatgctcaagatcaaaactttatagaacagtcaacattttagattcggtaaccttttttttcttccaattataatctctgcttctagccacttccgccagcagttggtggaagacttactaggtgcagggcactttccaagttcatcacaacaacctgcttgttttcatgagacaataatccgaaaagttcgctttgatatattcctggagggccaagcccatctatttacaaaaggtgaacagcaaaatcaagcactgctttatgggcaggaacacaagagaaagcaaactgcccaagaagtcatcatgtcagaaactcaatctcaacaaaataatttccatcagggaacttcagggtttcttgggggcttatgagtctcaccggtcaacccaggaggcctcactacaagagccttgacaaggcactgttttttgtgggactgggagttcacactgatgaagcaaacctttgaatttttgcacagctcttgtcagaaagccctgagttccccctggataaagagttaattttaatccttccctataattatacttcaaaatatttgacatctgctattatgccttctttagatctttcttctgcggtgcagacatttctagtaagtgtttgactacttgtatggcattagctttcacagaaaattgtttcacttaaaactgtggattggcctaggctaaggacaaaaataaactaagtacctgtagtgtatttatgtgatatgtgtcaagttactcaaagttattgctgttggaactgaacaataatatttcccagatagctggccttagcatgtgatcacggttgttgtatttttaatttttgtcttttacagtatgagaggtgtaggttaatttgtttatttcctataaatttgtatttatgtgtatataaaatgtacaatgaatgtaaatatgactttctggaaagtttagactacatttagaatctctattcaaaatcaaaatgctgctcaaatgaatttaaccaacatctaggtgcttaatttctcattttatcccacttatgagattgggaaaaagatcaatatgagaaataccatacagataccttaaatgtatgcatttgtgcaacaatttttgagaaggtgagtggcaatttataatttagttggcaatttataatagaacttatagcttttaaaagactttttaaagacattaaatgtaaacttaaaaatgtttagatcttgtttcaaactttacaatagcattcttcaaaatattaagttatatattttataggcatttagttgcttattaaaagcactgattttcaaactttttgatttaagaacaattatttaagatcgtctcagaagatgggatcttcgtttcaagaaaagggaatcaagtttgcctttgagataatacgttacactaagaaaaggaaaatgtggatagtaaaacccacctctctcatcctattgtactctcttctgctttttagaagcctgcacttaagcttagatttgtgaagggagagtagaaggggagaagtagaaccacagtgttttatttatttttctaaaactcttactaaatccagattttttaaactgttttaaatgtgaattcttcccagaaatttcaatgcattgcatatttagccttcggcatatttttcatgaatagatcatgaagtcataggcttccaaggcataggaagagatcttgcaggtctagtattttaataatgcactattacccagggcagatattatgagaaactgtttcttctctaagggtttatggcagactttgcttttttaacatgtgagaaatgaattttttattttgtgatttatgtgatttcttttgctgagtgaaggaaaggagaaattgttgctattgtcagcatcttaaaggtatttccagtcaaggcaaggctaagtgctttgtgatagtattaagcaagtcatgttttgaatggattacctgtagtgactcattggaatgatataattatacaagtaatgccaaaaaccaagtcaaagcctaattaaccaaagcactcatttaaaaatcatcatgtttggacctatctggacctctcagcactgtaaaatagttttggttttgtggcatatgaatagctgtttaacaaatcaaagttagctttttgcttctcagcttttttgggcaatacaagttaagttcttaatggggagacattatcatggcatgacttaagggaacattggtttgtgaaggaaaaacagattatctaaagccatctctatgtttctgttcagataaagattaatgagttctgtgtttatatcagctttgtatatttcatcttagccattctatcctagaaagattttaatgtgagcttaagatgtaaataaataattttgcaaacatgaaaaaaaaaaaaaaaaa

SEQ ID NO: 6 - protein, 2139 aa, Homo sapiens
MAEVTVPRVYVVFGIHCIMAKASSDVQVSGFHRKIQHVKNELCHMLSLEEVAPVLQQTLLQDNLLGRVHFDQFKEALILILSRTLSNEEHFQEPDCSLEAQPKYVRGGKRYGRRSLPEFQESVEEFPEVTVIEPLDEEARPSHIPAGDCSEHWKTQRSEEYEAEGQLRFWNPDDLNASQSGSSPPQDWIEEKLQEVCEDLGITRDGHLNRKKLVSICEQYGLQNVDGEMLEEVFHNLDPDGTMSVEDFFYGLFKNGKSLTPSASTPYRQLKRHLSMQSFDESGRRTTTSSAMTSTIGFRVFSCLDDGMGHASVERILDTWQEEGIENSQEILKALDFSLDGNINLTELTLALENELLVTKNSIHQAALASFKAEIRHLLERVDQVVREKEKLRSDLDKAEKLKSLMASEVDDHHAAIERRNEYNLRKLDGEYKERIAALKNELRKEREQILQQAGKQRLELEQEIEKAKTEENYIRDRLALSLKENSRLENELLENAEKLAEYENLTNKLQRNLENVLAEKFGDLDPSSAEFFLQEERLTQMRNEYERQCRVLQDQVDELQSELEEYRAQGRVLRLPLKNSPSEEVEANSGGIEPEHGLGSEECNPLNMSIEAELVIEQMKEQHHRDICCLRLELEDKVRHYEKQLDETVVSCKKAQENMKQRHENETRTLEKQISDLKNEIAELQGQAAVLKEAHHEATCRHEEEKKQLQVKLEEEKTHLQEKLRLQHEMELKARLTQAQASFEREREGLQSSAWTEEKVRGLTQELEQFHQEQLTSLVEKHTLEKEELRKELLEKHQRELQEGREKMETECNRRTSQIEAQFQSDCQKVTERCESALQSLEGRYRQELKDLQEQQREEKSQWEFEKDELTQECAEAQELLKETLKREKTTSLVLTQEREMLEKTYKEHLNSMVVERQQLLQDLEDLRNVSETQQSLLSDQILELKSSHKRELREREEVLCQAGASEQLASQRLERLEMEHDQERQEMMSKLLAMENIHKATCETADRERAEMSTEISRLQSKIKEMQQATSPLSMLQSGCQVIGEEEVEGDGALSLLQQGEQLLEENGDVLLSLQRAHEQAVKENVKMATEISRLQQRLQKLEPGLVMSSCLDEPATEFFGNTAEQTEQFLQQNRTKQVEGVTRRHVLSDLEDDEVRDLGSTGTSSVQRQEVKIEESEASVEGFSELENSEETRTESWELKNQISQLQEQLMMLCADCDRASEKKQDLLFDVSVLKKKLKMLERIPEASPKYKLLYEDVSRENDCLQEELRMMETRYDEALENNKELTAEVFRLQDELKKMEEVTETFLSLEKSYDEVKIENEGLNVLVLRLQGKIEKLQESVVQRCDCCLWEASLENLEIEPDGNILQLNQTLEECVPRVRSVHHVIEECKQENQYLEGNTQLLEKVKAHEIAWLHGTIQTHQERPRVQNQVILEENTTLLGFQDKHFQHQATIAELELEKTKLQELTRKLKERVTILVKQKDVLSHGEKEEELKAMMHDLQITCSEMQQKVELLRYESEKLQQENSILRNEITTLNEEDSISNLKLGTLNGSQEEMWQKTETVKQENAAVQKMVENLKKQISELKIKNQQLDLENTELSQKNSQNQEKLQELNQRLTEMLCQKEKEPGNSALEEREQEKFNLKEELERCKVQSSTLVSSLEAELSEVKIQTHIVQQENHLLKDELEKMKQLHRCPDLSDFQQKISSVLSYNEKLLKEKEALSEELNSCVDKLAKSSLLEHRIATMKQEQKSWEHQSASLKSQLVASQEKVQNLEDTVQNVNLQMSRMKSDLRVTQQEKEALKQEVMSLHKQLQNAGGKSWAPEIATHPSGLHNQQKRLSWDKLDHLMNEEQQLLWQENERLQTMVQNTKAELTHSREKVRQLESNLLPKHQKHLNPSGTMNPTEQEKLSLKRECDQFQKEQSPANRKVSQMNSLEQELETIHLENEGLKKKQVKLDEQLMEMQHLRSTATPSPSPHAWDLQLLQQQACPMVPREQFLQLQRQLLQAERINQHLQEELENRTSETNTPQGNQEQLVTVMEERMIEVEQKLKLVKRLLQEKVNQLKEQLCKNTKADAMVKDLYVENAQLLKALEVTEQRQKTAEKKNYLLEEKIASLSNIVRNLTPAPLTSTPPLRS

SEQ ID NO: 7 - DNA, 3768 bp, Homo sapiens
  CDS (77)..(3766)
gccttggattttcaggttttcatcctgatacttgtttacttttctggggcagaaaagcttgcactaattgctctccatggtggctaattttttcaagagcttgattttaccttacattcataagctttgcaaaggaatgtttacaaagaaattgggaaatacaaacaaaaacaaagagtatcgtcagcagaaaaaggatcaagacttccccactgctggccagaccaaatcccccaaattttcttacacttttaaaagcactgtaaagaagattgcaaagtgttcatccactcacaacttatccactgaggaagacgaggccagtaaagagttttccctctcaccaacattcagttaccgagtagctattgccaatggcctacaaaagaatgctaaagtaaccaccagtgataatgaggatctgcttcaagagctctcttcaatcgagagttcctactcagaatcattaaatgaactaaggagtagcacagaaaaccaggcacaatcaacacacacaatgccagttagacgcaacagaaagagttcaagcagccttgcaccctctgagggcagctctgacggggagcgtactctacatggcttaaaactgggagctttacgaaaactgagaaaatggaaaaagagtcaagaatgtgtctcctcagactcagagttaagcaccatgaaaaaatcctggggaataagaagtaagtctttggacagaactgtccgaaacccaaagacaaatgccctggagccagggttcagttcctctggctgcattagccaaacacatgatgtcatggaaatgatctttaaggaacttcagggaataagtcagattgaaacagaactttctgaactacgagggcacgtcaatgctctcaagcactccatcgatgagatctccagcagtgtggaggttgtacaaagtgaaattgagcagttgcgcacagggtttgtccagtctcggagggaaactagagacatccatgattatattaagcacttaggtcatatgggtagcaaggcaagcctgagatttttaaatgtgactgaagaaagatttgaatatgttgaaagcgtggtgtaccaaattctaatagataaaatgggtttttcagatgcaccaaatgctattaaaattgaatttgctcagaggataggacaccagagagactgcccaaatgcaaagcctcgacccatacttgtgtactttgaaacccctcaacaaagggattctgtcttaaaaaagtcatataaactcaaaggaacaggcattggaatctcaacagatattctaactcatgacatcagagaaagaaaagagaaagggataccatcctcccagacatatgagagcatggctataaagttgtctactccagagccaaaaatcaagaagaacaattggcagtcacctgatgacagtgatgaagatcttgaatctgacctcaatagaaacagttacgctgtgctttccaagtcagagcttctaacaaagggaagtacttccaagccaagctcaaaatcacacagtgctagatccaagaataaaactgctaatagcagcagaatttcaaataaatcagattatgataaaatctcctcacagttgccagaatcagatatcttggaaaagcaaaccacaacccattatgcagatgcaacacctctctggcactcacagagtgattttttcactgctaaacttagtcgttctgaatcagatttttccaaattgtgtcagtcttactcagaagatttttcagaaaatcagtttttcactagaactaatggaagctctctcctgtcatcttcggaccgggagctatggcagaggaaacaggaaggaacagcgaccctgtatgacagtcccaaggaccagcatttgaatggaagtgttcagggtatccaagggcagactgaaactgaaaacacagaaactgtggatagtggaatgagtaatggcatggtgtgtgcatctggagaccggagtcattacagtgattctcagctctctttacatgaggatctttctccatggaaggaatggaatcaaggagctgatttaggcttggattcatccacccaggaaggttttgattatgaaacaaacagtctttttgaccaacagcttgatgtttacaataaagacctagaatacttgggaaagtgccacagtgatcttcaagatgactcagagagctacgacttaactcaagatgacaattcttctccatgccctggcttggataatgaaccacaaggccagtgggttggccaatatgattcttatcagggagctaattctaatgagctataccaaaatcaaaaccagttgtccatgatgtatcgaagtcaaagtgaattgcaaagtgatgattcagaggatgccccacccaaatcatggcatagtcgattaagcattgacctttctgataagactttcagcttcccaaaatttggatctacactgcagagggctaaatcagccttggaagtagtatggaacaaaagcacacagagtctgagtgggtatgaggacagtggctcttcattaatggggagatttcggacattatctcaatcaactgcaaatgagtcaagtaccacacttgactctgatgtctacacggagccctattactataaagcagaggatgaggaagattatactgaaccagtggctgacaatgaaacagattatgttgaagtcatggaacaagtccttgctaaactagaaaacaggactagtattactgaaacagatgaacaaatgcaagcatatgatcacctttcatatgaaacaccttatgaaaccccacaagatgagggttatgatggtccagcagatgatatggttagtgaagaggggttagaacccttaaatgaaacatcagctgagatggaaataagagaagatgaaaaccaaaacattcctgaacagccagtggagatcacaaagccaaagagaattcgtccttctttcaaagaagcagctttaagggcctataaaaagcaaatggcagagttggaagagaagatcttggctggagatagcagttctgtggatgaaaaggctcgaatagtaagtggcaatgatttggatgcttccaaattttctgcactccaggtgtgtggtggggctggaggtggactttatggtattgacagcatgccggatcttcgcagaaaaaaaactttgcctattgtccgagatgtggccatgaccctggctgcccggaaatctggactctccctggctatggtgattaggacatccctaaataatgaggaactgaaaatgcacgtcttcaagaagaccttgcaggcactgatctaccctatgtcttctaccatcccacacaattttgaggtctggacggctaccacacccacctactgttatgagtgtgaagggctcctgtggggcattgcaaggcaaggcatgaagtgtctggagtgtggagtgaaatgccacgaaaagtgtcaggacctgctaaacgctgactgcttgcagagagcagcagaaaagagttctaaacatggtgccgaagacaagactcagaccattattacagcaatgaaagaaagaatgaagatcagggagaaaaaccggccagaagtatttgaagtaatccaggaaatgtttcagatttctaaagaagattttgtgcagtttacaaaggcggccaaacagagtgtactggatgggacatctaagtggtctgcaaaaataaccatcacagtggtttctgcacaagg

SEQ ID NO: 8 - protein, 1230 aa, Homo sapiens
MVANFFKSLILPYIHKLCKGMFTKKLGNTNKNKEYRQQKKDQDFPTAGQTKSPKFSYTFKSTVKKIAKCSSTHNLSTEEDEASKEFSLSPTFSYRVAIANGLQKNAKVTTSDNEDLLQELSSIESSYSESLNELRSSTENQAQSTHTMPVRRNRKSSSSLAPSEGSSDGERTLHGLKLGALRKLRKWKKSQECVSSDSELSTMKKSWGIRSKSLDRTVRNPKTNALEPGFSSSGCISQTHDVMEMIFKELQGISQIETELSELRGHVNALKHSIDEISSSVEVVQSEIEQLRTGFVQSRRETRDIHDYIKHLGHMGSKASLRFLNVTEERFEYVESVVYQILIDKMGFSDAPNAIKIEFAQRIGHQRDCPNAKPRPILVYFETPQQRDSVLKKSYKLKGTGIGISTDILTHDIRERKEKGIPSSQTYESMAIKLSTPEPKIKKNNWQSPDDSDEDLESDLNRNSYAVLSKSELLTKGSTSKPSSKSHSARSKNKTANSSRISNKSDYDKISSQLPESDILEKQTTTHYADATPLWHSQSDFFTAKLSRSESDFSKLCQSYSEDFSENQFFTRTNGSSLLSSSDRELWQRKQEGTATLYDSPKDQHLNGSVQGIQGQTETENTETVDSGMSNGMVCASGDRSHYSDSQLSLHEDLSPWKEWNQGADLGLDSSTQEGFDYETNSLFDQQLDVYNKDLEYLGKCHSDLQDDSESYDLTQDDNSSPCPGLDNEPQGQWVGQYDSYQGANSNELYQNQNQLSMMYRSQSELQSDDSEDAPPKSWHSRLSIDLSDKTFSFPKFGSTLQRAKSALEVVWNKSTQSLSGYEDSGSSLMGRFRTLSQSTANESSTTLDSDVYTEPYYYKAEDEEDYTEPVADNETDYVEVMEQVLAKLENRTSITETDEQMQAYDHLSYETPYETPQDEGYDGPADDMVSEEGLEPLNETSAEMEIREDENQNIPEQPVEITKPKRIRPSFKEAALRAYKKQMAELEEKILAGDSSSVDEKARIVSGNDLDASKFSALQVCGGAGGGLYGIDSMPDLRRKKTLPIVRDVAMTLAARKSGLSLAMVIRTSLNNEELKMHVFKKTLQALIYPMSSTIPHNFEVWTATTPTYCYECEGLLWGIARQGMKCLECGVKCHEKCQDLLNADCLQRAAEKSSKHGAEDKTQTIITAMKERMKIREKNRPEVFEVIQEMFQISKEDFVQFTKAAKQSVLDGTSKWSAKITITVVSAQ